(19) United States
(12) Patent Application Publication
(10) Pub. No.: US 2025/0101534 A1
(43) Pub. Date: Mar. 27, 2025

(54) USE OF SPOP MUTATIONS AS A PREDICTIVE BIOMARKER

(71) Applicant: Foundation Medicine, Inc., Boston, MA (US)

(72) Inventor: Ryon P. GRAF, Encinitas, CA (US)

(73) Assignee: Foundation Medicine, Inc., Boston, MA (US)

(21) Appl. No.: 18/920,014

(22) Filed: Oct. 18, 2024

Related U.S. Application Data
(63) Continuation of application No. PCT/US2023/019792, filed on Apr. 25, 2023.
(60) Provisional application No. 63/335,184, filed on Apr. 26, 2022.

(51) Int. Cl. C12Q 1/6886; A61K 31/4166; A61K 31/4178; A61K 31/4439; A61K 31/58; A61K 45/06; A61P 35/04; C12Q 1/6806; C12Q 1/6855; C12Q 1/6869

(52) U.S. Cl. CPC C12Q 1/6886; A61K 31/4166; A61K 31/4178; A61K 31/4439; A61K 31/58; A61K 45/06; A61P 35/04; C12Q 1/6806; C12Q 1/6855; C12Q 1/6869; C12Q 2600/106; C12Q 2600/156

(57) ABSTRACT

The present disclosure provides biomarkers associated with response to a treatment in individuals having prostate cancer, as well as to methods of diagnosis, assessment, and treatment of prostate cancer.

### CROSS-REFERENCE TO RELATED APPLICATIONS

This application is a continuation application of International Application No. PCT/US2023/019792, filed internationally on Apr. 25, 2023, which claims the priority benefit of U.S. Provisional Patent Application Ser. No. 63/335,184, filed Apr. 26, 2022, the disclosures of each of which are hereby incorporated by reference in their entirety.

### FIELD

The present disclosure relates to biomarkers associated with response to treatments in individuals having prostate cancer, as well as to methods of diagnosis, assessment, and treatment of prostate cancer.

### BACKGROUND

Prostate cancer is one of the most common cancer types. While most localized prostate cancer can be cured with surgery, progression of the disease into a metastatic state presents additional treatment challenges (FIG. 4). Intensification of androgen deprivation therapy (ADT) with taxanes (e.g., docetaxel) or novel hormonal therapy (NHT) is the current standard of care recommended for patients with metastatic prostate cancer, including metastatic hormone-sensitive prostate cancer (mHSPC). While the combination of ADT with docetaxel or NHT have shown superiority over the use of single agents, there is a lack of available tools to predict which treatment would be better suited for a particular patient. For instance, the National Comprehensive Cancer Network (NCCN) does not currently recommend any biomarkers to help predict the success of initial systemic therapy in mHSPC.

Thus, there is a need for predictive biomarkers that could be used to select a treatment for metastatic prostate cancer patients.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY

In one aspect, provided herein is a method of identifying an individual having a metastatic hormone-sensitive prostate cancer (mHSPC) or a non-metastatic castration-resistant prostate cancer (nmCRPC) who may benefit from a treatment comprising hormonal therapy, the method comprising detecting in a sample from the individual: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, wherein the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising the hormonal therapy.

In yet another aspect, provided herein is a method of selecting a treatment for an individual having a mHSPC or a nmCRPC, the method comprising detecting in a sample from the individual: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, wherein the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising a hormonal therapy.

In yet another aspect, provided herein is a method of identifying one or more treatment options for an individual having a mHSPC or a nmCRPC, the method comprising: (a) detecting in a sample from the individual (i) one or more mutations in an SPOP gene, or an SPOP polypeptide encoded by the mutated SPOP gene, and/or (ii) a TMPRSS2-ERG fusion nucleic acid molecule comprising a fusion between a TMPRSS2 gene, or a portion thereof, and an ERG gene, or a portion thereof; and (b) generating a report comprising one or more treatment options identified for the individual based at least in part on detection of the one or more mutations in the SPOP gene, or the SPOP polypeptide encoded by the mutated SPOP gene, and/or the TMPRSS2-ERG fusion nucleic acid molecule in the sample, wherein the one or more treatment options comprise a hormonal therapy.

In yet another aspect, provided herein is a method of selecting a treatment for an individual having a mHSPC or a nmCRPC, comprising acquiring knowledge of (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, in a sample from the individual, wherein responsive to said knowledge, the individual is classified as a candidate to receive a treatment comprising a hormonal therapy.

In yet another aspect, provided herein is a method of treating or delaying progression of a mHSPC or a nmCRPC, comprising: (a) acquiring knowledge of (i) one or more mutations in an SPOP gene, or an SPOP polypeptide encoded by the mutated SPOP gene, and/or (ii) a TMPRSS2-ERG fusion nucleic acid molecule comprising a fusion between a TMPRSS2 gene, or a portion thereof, and an ERG gene, or a portion thereof, in a sample from an individual; and (b) administering to the individual an effective amount of a hormonal therapy responsive to said knowledge.

In yet another aspect, provided herein is a method of predicting survival of an individual having a mHSPC or a nmCRPC, comprising acquiring knowledge of: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, in a sample from the individual. In some embodiments, responsive to said knowledge, the individual is predicted to have longer survival when treated with an a hormonal therapy, as compared to an individual whose cancer does not comprise the one or more mutations in the SPOP gene, or the SPOP polypeptide encoded by the mutated SPOP gene, and/or the TMPRSS2-ERG fusion nucleic acid. In other embodiments, responsive to said knowledge, the individual is predicted to have a higher likelihood of survival when treated with an a hormonal therapy as compared to a treatment with a taxane. In some embodiments, the survival is an overall survival, a progression-free survival, a disease-free survival, an objective response rate, a time to tumor progression, a time to treatment failure, a durable complete response, a time to cancer-resistant pancreatic cancer progression, or a time to next treatment.

In some embodiments of any of the methods disclosed herein, the one or more mutations in the SPOP gene are one or more of a base substitution, a short insertion/deletion (indel), a copy number alteration, or a genomic rearrangement. In some embodiments, the one or more mutations in the SPOP gene comprise one or more of a 305T>G mutation, a 397T>G mutation, a 399C>G mutation, a 393G>C mutation, a 304T>G mutation, a 374T>G mutation, a 398T>C mutation, a 398T>G mutation, a 259T>A mutation, a 260A>G mutation, a 304T>A mutation, a 356G>A mutation, a 375T>G mutation, a 389A>T mutation, a 391T>C mutation, a 391T>G mutation, a 393G>T mutation, a 397T>A mutation, a 399C>A mutation, or any combination thereof. In some embodiments, the one or more mutations in the SPOP gene result in one or more amino acid substitutions in an SPOP polypeptide encoded by the mutated SPOP gene, wherein the one or more amino acid substitutions correspond to one or more of R45W, R70*, Y87C, Y87N, Y87S, Y87F, Y87D, F102C, F102V, F102S, F102I, F102L, F104V, F102Y, F104C, F104S, F104I, A116G, M117V, S119N, F125I, F125V, F125L, F125C, K129E, D130V, D130N, W131G, W131C, W131R, W131L, W131S, F133L, F133V, F133I, F133C, F133S, K134N, A277V, and L282R, or any combination thereof. In some embodiments, the one or more mutations in the SPOP gene result in decreased activity of an SPOP polypeptide encoded by the mutated SPOP gene. In some embodiments, the SPOP polypeptide encoded by the mutated SPOP gene has decreased activity as compared to an SPOP polypeptide encoded by an SPOP gene that does not comprise the one or more mutations. In some embodiments, the SPOP polypeptide encoded by the mutated SPOP gene is oncogenic. In some embodiments, the SPOP polypeptide encoded by the mutated SPOP gene promotes cancer cell survival, angiogenesis, cancer cell proliferation, or any combination thereof.

In some embodiments of any of the methods disclosed herein, the TMPRSS2-ERG fusion nucleic acid molecule encodes a TMPRSS2-ERG fusion polypeptide. In some embodiments, the TMPRSS2-ERG fusion polypeptide encoded by the TMPRSS2-ERG fusion nucleic acid molecule is oncogenic. In some embodiments, the TMPRSS2-ERG fusion polypeptide encoded by the TMPRSS2-ERG fusion nucleic acid molecule promotes cancer cell survival, angiogenesis, cancer cell proliferation, and any combination thereof.

In some embodiments, of any of the methods disclosed herein, the hormonal therapy is a novel hormonal therapy (NHT).

In some embodiments of any of the methods disclosed herein, the treatment further comprises administering to the individual an anti-androgen agent. In some embodiments of any of the methods disclosed herein, the hormonal therapy is a second generation hormonal therapy. In some embodiments, the hormonal therapy comprises one or more of abiraterone, enzalutamide, apalutamide, and darolutamide, or any combination thereof. In some embodiments, the hormonal therapy treatment further comprises an androgen deprivation therapy (ADT). In some embodiments, the ADT comprises one or more of an orchiectomy, a LHRH agonist, a LHRH antagonist, an anti-androgen agent, an estrogen, and an androgen synthesis inhibitor, or any combination thereof. In some embodiments, the orchiectomy is a bilateral orchiectomy. In some embodiments, the LHRH agonist is goserelin acetate, histrelin acetate, leuprolide acetate, or triptorelin acetate. In some embodiments, the LHRH antagonist is degarelix. In some embodiments, the anti-androgen agent is bicalutamide, flutamide, nilutamide, enzalutamide, apalutamide, or darolutamide. In some embodiments, the estrogen is diethylstilbestrol. In some embodiments, the androgen synthesis inhibitor is abiraterone or ketoconazole. In some embodiments of any of the methods disclosed herein, the treatment does not comprise a chemotherapy. In some embodiments, the chemotherapy is a taxane. In some embodiments, the taxane is docetaxel, cabazitaxel, or paclitaxel.

In some embodiments of any of the methods disclosed herein, the mHSPC is a de novo mHSPC or a recurrent mHSPC. In some embodiments, the individual having a mHSPC or nmCRPC is at risk of progressing to a metastatic castration-resistant prostate cancer (mCRPC).

In some embodiments of any of the methods disclosed herein, the individual has received a prior anti-cancer treatment, or is being treated with an anti-cancer treatment. In some embodiments, the prior anti-cancer treatment comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the methods disclosed herein, the cancer has not been previously treated.

In some embodiments of any of the methods disclosed herein, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the methods disclosed herein, the sample comprises a tissue biopsy sample or a liquid biopsy sample. In some embodiments, the sample is a tissue biopsy and comprises a tumor biopsy, tumor specimen, or circulating tumor cells. In some embodiments, the sample is a liquid biopsy sample and comprises blood, serum, plasma, cerebrospinal fluid, sputum, stool, urine, or saliva. In some embodiments, the sample comprises cells and/or nucleic acids from the cancer.

In some embodiments, the sample comprises mRNA, DNA, circulating tumor DNA (ctDNA), cell-free DNA, cell-free RNA from the cancer, or any combination thereof. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the method further comprising obtaining the sample from the individual. In some embodiments, the sample comprises cancer cells. In some embodiments, the acquiring knowledge of the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid comprises detecting the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid in the sample.

In some embodiments of any of the methods disclosed herein, the one or more mutations in the SPOP gene and/or the TMPRSS2-ERG fusion nucleic acid are detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS). In some embodiments, the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample. In some embodiments, the adapters comprise one or more of amplification primer sequences, flow cell adapter hybridization sequences, unique molecular identifier sequences, substrate adapter sequences, or sample index sequences. In some embodiments, amplifying nucleic acid molecules comprises performing a polymerase chain reaction (PCR) technique, a non-PCR amplification technique, or an isothermal amplification technique. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin. In some embodiments of any of the methods disclosed herein, the SPOP polypeptide encoded by the mutated SPOP gene and/or the TMPRSS2-ERG fusion polypeptide encoded by the TMPRSS2-ERG fusion nucleic acid are detected in the sample by one or more of: immunoblotting, enzyme linked immunosorbent assay (ELISA), immunohistochemistry, or mass spectrometry.

In some embodiments of any of the methods disclosed herein, the individual is a human.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIGS. 1A-1B show a schematic of the cohort selection process. FIG. 1A shows a schematic of the cohort inclusion criteria and study outcomes. FIG. 1B shows a schematic of the selection of a cohort of patients for study inclusion. Out of 3783 patients screened from a de-identified clinic-genomics database, 401 patients met the criteria for cohort inclusion. As shown in the figures: Dx, diagnosis; Tx, treatment; NHT, novel hormone therapy; CRPC, castration-resistant prostate cancer; CGP, comprehensive genomic profiling; mCRPC, metastatic castration-resistant prostate cancer; ICPI, immune checkpoint inhibitor; TTNT, time to next therapy; OS, overall survival; mut, mutated; wt, wild-type, and PSA, prostate-specific antigen.

FIGS. 2A-2D show the outcomes for SPOP-wild-type or SPOP-mutated metastatic hormone-sensitive prostate cancer (mHSPC) patients treated with novel hormonal therapy (NHT) or docetaxel. FIGS. 2A-2B show the time to castration-resistant prostate cancer (CRPC) (FIG. 2A) and overall survival (OS) (FIG. 2B) of SPOP-wild-type and SPOP-mutated mHSPC patients treated with NHT. FIGS. 2C-2D show the time to CRPC (FIG. 2C) and overall survival (OS) (FIG. 2D) of SPOP-wild-type and SPOP-mutated mHSPC patients treated with docetaxel. The results demonstrate that, compared to SPOP-wild-type mHSPC patients, SPOP-mutated mHSPC patients had improved time to CRPC and OS when treated with NHT. However, compared to SPOP-wild-type mHSPC patients, SPOP-mutated mHSPC patients had similar time to CRPC and OS when treated with docetaxel. As shown in the figure: time to CRPC, time to castration resistant prostate cancer; OS, overall survival; HR, hazard ratio; 95% CI, 95% confidence interval, SPOP mut, SPOP-mutated; and SPOP wt, SPOP-wild-type.

FIGS. 3A-3D show the outcomes for TMPRSS2-ERG fusion positive or TMPRSS2-ERG fusion negative metastatic hormone-sensitive prostate cancer (mHSPC) patients treated with a novel hormonal therapy (NHT) or docetaxel. FIGS. 3A-3B show the time to castration-resistant prostate cancer (CRPC) (FIG. 3A) and overall survival (OS) (FIG. 3B) of TMPRSS2-ERG fusion positive or TMPRSS2-ERG fusion negative mHSPC patients treated with NHT. FIGS. 3C-3D show the time to CRPC (FIG. 3C) and overall survival (OS) (FIG. 3D) of TMPRSS2-ERG fusion positive or TMPRSS2-ERG fusion negative mHSPC patients treated with docetaxel. The results demonstrate that, compared to TMPRSS2-ERG fusion negative mHSPC patients, TMPRSS2-ERG fusion positive mHSPC patients had improved time to CRPC and OS when treated with a NHT. However, compared to TMPRSS2-ERG fusion negative mHSPC patients, compared to TMPRSS2-ERG fusion positive mHSPC patients had similar time to CRPC and OS when treated with docetaxel. As shown in the figure: time to CRPC, time to castration resistant prostate cancer; OS, overall survival; HR, hazard ratio; 95% CI, 95% confidence interval, TMPRSS2-ERF (+), TMPRSS2-ERG fusion positive; and TMPRSS2-ERG (−), TMPRSS2-ERG fusion negative.

FIG. 4 shows a schematic of prostate cancer disease states and progression. As shown in the figure: PC, prostate cancer; and PSA, prostate-specific antigen.

### DETAILED DESCRIPTION

In one aspect, provided herein are methods of identifying an individual having a metastatic hormone-sensitive prostate cancer (mHSPC) or a non-metastatic castration-resistant prostate cancer (nmCRPC) who may benefit from a treatment comprising a hormonal therapy, the method comprising detecting in a sample from the individual one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof. In some embodiments, the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising the hormonal therapy. In some embodiments, the hormonal therapy is a novel hormonal therapy (NHT).

In another aspect, provided herein are methods of selecting a treatment for an individual having mHSPC or nmCRPC comprising detecting in a sample from the individual one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof. In some embodiments, the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising a hormonal therapy. In some embodiments, the hormonal therapy is a NHT.

In yet another aspect, provided herein are methods of identifying one or more treatment options for an individual having a mHSPC or nmCRPC comprising detecting in a sample from the individual one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof; and generating a report comprising one or more treatment options identified for the individual based at least in part on detection of the one or more mutations in the SPOP gene, or the SPOP polypeptide encoded by the mutated SPOP gene, and/or the TMPRSS2-ERG fusion nucleic acid molecule in the sample, wherein the one or more treatment options comprise a hormonal therapy. In some embodiments, the hormonal therapy is a NHT.

In yet another aspect, provided herein are methods of selecting a treatment for an individual having a mHSPC or nmCRPC, comprising acquiring knowledge of one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof, in a sample from the individual. In some embodiments, responsive to said knowledge, the individual is classified as a candidate to receive a treatment comprising a NHT. In some embodiments, the hormonal therapy is a NHT.

In yet another aspect, provided herein are methods of treating or delaying progression of mHSPC or nmCRPC comprising acquiring knowledge of one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof, in a sample from an individual; and administering to the individual an effective amount of a hormonal therapy responsive to said knowledge. In some embodiments, the hormonal therapy is a NHT.

In yet another aspect, provided herein are methods of predicting survival of an individual having a mHSPC or nmCRPC, comprising acquiring knowledge of one or more mutations in an SPOP gene, or an SPOP polypeptide encoded by the mutated SPOP gene, and/or a TMPRSS2-ERG fusion nucleic acid molecule comprising a fusion between a TMPRSS2 gene, or a portion thereof, and an ERG gene, or a portion thereof, in a sample from the individual. In some embodiments, responsive to said knowledge, the individual is predicted to have longer survival when treated with a hormonal therapy, as compared to an individual whose cancer does not comprise the one or more mutations in the SPOP gene, or the SPOP polypeptide encoded by the mutated SPOP gene, and/or the TMPRSS2-ERG fusion nucleic acid. In other embodiments, responsive to said knowledge, the individual is predicted to have a higher likelihood of survival when treated with a hormonal therapy as compared to a treatment with a taxane. In some embodiments, the survival is an overall survival, a progression-free survival, a disease-free survival, an objective response rate, a time to tumor progression, a time to treatment failure, a durable complete response, a time to cancer-resistant pancreatic cancer progression, or a time to next treatment. In some embodiments, the hormonal therapy is a NHT.

### I. Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

The term “detection” includes any means of detecting, including direct and indirect detection. The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy, e.g., a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations, or other mutations or alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

“Subject response” or “response” can be assessed using any endpoint indicating a benefit to the subject, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a subject or a subject's “responsiveness” to treatment with a medicament and similar wording refers to the clinical or therapeutic benefit imparted to a subject at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

As used herein, “treatment” (and grammatical variations thereof such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the subject herein is a human.

As used herein, “administering” is meant a method of giving a dosage of an agent or a pharmaceutical composition (e.g., a pharmaceutical composition including the agent) to a subject. Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The terms “concurrently” or “in combination” are used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

“Acquire” or “acquiring” as the terms are used herein, refer to obtaining possession of a physical entity, or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

“Acquiring a sequence” or “acquiring a read” as the term is used herein, refers to obtaining possession of a nucleotide sequence or amino acid sequence, by “directly acquiring” or “indirectly acquiring” the sequence or read. “Directly acquiring” a sequence or read means performing a process (e.g., performing a synthetic or analytical method) to obtain the sequence, such as performing a sequencing method (e.g., a Next-generation Sequencing (NGS) method). “Indirectly acquiring” a sequence or read refers to receiving information or knowledge of, or receiving, the sequence from another party or source (e.g., a third-party laboratory that directly acquired the sequence). The sequence or read acquired need not be a full sequence, e.g., sequencing of at least one nucleotide, or obtaining information or knowledge, that identifies one or more of the alterations disclosed herein as being present in a sample, biopsy or subject constitutes acquiring a sequence.

Directly acquiring a sequence or read includes performing a process that includes a physical change in a physical substance, e.g., a starting material, such as a sample described herein. Exemplary changes include making a physical entity from two or more starting materials, shearing or fragmenting a substance, such as a genomic DNA fragment; separating or purifying a substance (e.g., isolating a nucleic acid sample from a tissue); combining two or more separate entities into a mixture, performing a chemical reaction that includes breaking or forming a covalent or non-covalent bond. Directly acquiring a value includes performing a process that includes a physical change in a sample or another substance as described above. The size of the fragment (e.g., the average size of the fragments) can be 2500 bp or less, 2000 bp or less, 1500 bp or less, 1000 bp or less, 800 bp or less, 600 bp or less, 400 bp or less, or 200 bp or less. In some embodiments, the size of the fragment (e.g., cfDNA) is between about 150 bp and about 200 bp (e.g., between about 160 bp and about 170 bp). In some embodiments, the size of the fragment (e.g., DNA fragments from liquid biopsy samples) is between about 150 bp and about 250 bp. In some embodiments, the size of the fragment (e.g., cDNA fragments obtained from RNA in liquid biopsy samples) is between about 100 bp and about 150 bp.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

“Alteration” or “altered structure” as used herein, of a gene or gene product (e.g., a marker gene or gene product) refers to the presence of a mutation or mutations within the gene or gene product, e.g., a mutation, which affects integrity, sequence, structure, amount or activity of the gene or gene product, as compared to the normal or wild-type gene. The alteration can be in amount, structure, and/or activity in a cancer tissue or cancer cell, as compared to its amount, structure, and/or activity, in a normal or healthy tissue or cell (e.g., a control), and is associated with a disease state, such as cancer. For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, epigenetic modification (e.g., methylation or acetylation status, or post-translational modification, in a cancer tissue or cancer cell, as compared to a normal, healthy tissue or cell. Exemplary mutations include, but are not limited to, point mutations (e.g., silent, missense, or nonsense), deletions, insertions, inversions, duplications, amplification, translocations, inter- and intra-chromosomal rearrangements. Mutations can be present in the coding or non-coding region of the gene. In certain embodiments, the alteration(s) is detected as a rearrangement, e.g., a genomic rearrangement comprising one or more introns or fragments thereof (e.g., one or more rearrangements in the 5′- and/or 3′-UTR). In certain embodiments, the alterations are associated (or not associated) with a phenotype, e.g., a cancerous phenotype (e.g., one or more of cancer risk, cancer progression, cancer treatment or resistance to cancer treatment). In one embodiment, the alteration (or tumor mutational burden) is associated with one or more of: a genetic risk factor for cancer, a positive treatment response predictor, a negative treatment response predictor, a positive prognostic factor, a negative prognostic factor, or a diagnostic factor.

As used herein, the term “indel” refers to an insertion, a deletion, or both, of one or more nucleotides in a nucleic acid of a cell. In certain embodiments, an indel includes both an insertion and a deletion of one or more nucleotides, where both the insertion and the deletion are nearby on the nucleic acid. In certain embodiments, the indel results in a net change in the total number of nucleotides. In certain embodiments, the indel results in a net change of about 1 to about 50 nucleotides.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

As used herein, the term “library” refers to a collection of nucleic acid molecules. In one embodiment, the library includes a collection of nucleic acid nucleic acid molecules, e.g., a collection of whole genomic, subgenomic fragments, cDNA, cDNA fragments, RNA, e.g., mRNA, RNA fragments, or a combination thereof. Typically, a nucleic acid molecule is a DNA molecule, e.g., genomic DNA or cDNA. A nucleic acid molecule can be fragmented, e.g., sheared or enzymatically prepared, genomic DNA. Nucleic acid molecules comprise sequence from a subject and can also comprise sequence not derived from the subject, e.g., an adapter sequence, a primer sequence, or other sequences that allow for identification, e.g., “barcode” sequences. In one embodiment, a portion or all of the library nucleic acid molecules comprises an adapter sequence. The adapter sequence can be located at one or both ends. The adapter sequence can be useful, e.g., for a sequencing method (e.g., an NGS method), for amplification, for reverse transcription, or for cloning into a vector. The library can comprise a collection of nucleic acid molecules, e.g., a target nucleic acid molecule (e.g., a tumor nucleic acid molecule, a reference nucleic acid molecule, or a combination thereof). The nucleic acid molecules of the library can be from a single subject. In embodiments, a library can comprise nucleic acid molecules from more than one subject (e.g., 2, 3, 4, 5, 6, 7, 8, 9, 10, 20, 30 or more subjects), e.g., two or more libraries from different subjects can be combined to form a library comprising nucleic acid molecules from more than one subject. In one embodiment, the subject is a human having, or at risk of having, a cancer or tumor.

“Complementary” refers to sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In certain embodiments, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In other embodiments, all nucleotide residues of the first portion are capable of base pairing with nucleotide residues in the second portion.

“Likely to” or “increased likelihood,” as used herein, refers to an increased probability that an item, object, thing or person will occur. Thus, in one example, a subject that is likely to respond to treatment has an increased probability of responding to treatment relative to a reference subject or group of subjects.

“Unlikely to” refers to a decreased probability that an event, item, object, thing or person will occur with respect to a reference. Thus, a subject that is unlikely to respond to treatment has a decreased probability of responding to treatment relative to a reference subject or group of subjects.

“Next-generation sequencing” or “NGS” or “NG sequencing” as used herein, refers to any sequencing method that determines the nucleotide sequence of either individual nucleic acid molecules (e.g., in single molecule sequencing) or clonally expanded proxies for individual nucleic acid molecules in a high throughput fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next-generation sequencing methods are known in the art, and are described, e.g., in Metzker, M. (2010) Nature Biotechnology Reviews 11:31-46, incorporated herein by reference. Next-generation sequencing can detect a variant present in less than 5% or less than 1% of the nucleic acids in a sample.

“Nucleotide value” as referred herein, represents the identity of the nucleotide(s) occupying or assigned to a nucleotide position. Typical nucleotide values include: missing (e.g., deleted); additional (e.g., an insertion of one or more nucleotides, the identity of which may or may not be included); or present (occupied); A; T; C; or G. Other values can be, e.g., not Y, wherein Y is A, T, G, or C; A or X, wherein X is one or two of T, G, or C; T or X, wherein X is one or two of A, G, or C; G or X, wherein X is one or two of T, A, or C; C or X, wherein X is one or two of T, G, or A; a pyrimidine nucleotide; or a purine nucleotide. A nucleotide value can be a frequency for 1 or more, e.g., 2, 3, or 4, bases (or other value described herein, e.g., missing or additional) at a nucleotide position. E.g., a nucleotide value can comprise a frequency for A, and a frequency for G, at a nucleotide position.

“Or” is used herein to mean, and is used interchangeably with, the term “and/or”, unless context clearly indicates otherwise. The use of the term “and/or” in some places herein does not mean that uses of the term “or” are not interchangeable with the term “and/or” unless the context clearly indicates otherwise.

A “control nucleic acid” or “reference nucleic acid” as used herein, refers to nucleic acid molecules from a control or reference sample. Typically, it is DNA, e.g., genomic DNA, or cDNA derived from RNA, not containing the alteration or variation in the gene or gene product. In certain embodiments, the reference or control nucleic acid sample is a wild-type or a non-mutated sequence. In certain embodiments, the reference nucleic acid sample is purified or isolated (e.g., it is removed from its natural state). In other embodiments, the reference nucleic acid sample is from a blood control, a normal adjacent tissue (NAT), or any other non-cancerous sample from the same or a different subject. In some embodiments, the reference nucleic acid sample comprises normal DNA mixtures. In some embodiments, the normal DNA mixture is a process matched control. In some embodiments, the reference nucleic acid sample has germline variants. In some embodiments, the reference nucleic acid sample does not have somatic alterations, e.g., serves as a negative control.

As used herein, “target nucleic acid molecule” refers to a nucleic acid molecule that one desires to isolate from the nucleic acid library. In one embodiment, the target nucleic acid molecules can be a tumor nucleic acid molecule, a reference nucleic acid molecule, or a control nucleic acid molecule, as described herein.

“Tumor nucleic acid molecule,” or other similar term (e.g., a “tumor or cancer-associated nucleic acid molecule”), as used herein refers to a nucleic acid molecule having sequence from a tumor cell. The terms “tumor nucleic acid molecule” and “tumor nucleic acid” may sometimes be used interchangeably herein. In one embodiment, the tumor nucleic acid molecule includes a subject interval having a sequence (e.g., a nucleotide sequence) that has an alteration (e.g., a mutation) associated with a cancerous phenotype. In other embodiments, the tumor nucleic acid molecule includes a subject interval having a wild-type sequence (e.g., a wild-type nucleotide sequence). For example, a subject interval from a heterozygous or homozygous wild-type allele present in a cancer cell. A tumor nucleic acid molecule can include a reference nucleic acid molecule. Typically, it is DNA, e.g., genomic DNA, or cDNA derived from RNA, from a sample. In certain embodiments, the sample is purified or isolated (e.g., it is removed from its natural state). In some embodiments, the tumor nucleic acid molecule is a cfDNA. In some embodiments, the tumor nucleic acid molecule is a ctDNA. In some embodiments, the tumor nucleic acid molecule is DNA from a CTC.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived. Moreover, an “isolated” nucleic acid molecule, such as an RNA molecule or a cDNA molecule, can be substantially free of other cellular material or culture medium, e.g., when produced by recombinant techniques, or substantially free of chemical precursors or other chemicals, e.g., when chemically synthesized.

### II. Methods of the Disclosure

In some aspects, provided herein are methods of treating or delaying progression of a metastatic hormone-sensitive prostate cancer (mHSPC) or a non-metastatic castration-resistant prostate cancer (nmCRPC) in an individual. In other aspects, provided herein are methods of selecting or identifying a treatment for or predicting survival of an individual having a mHSPC or a nmCRPC. In some embodiments, the methods provided herein comprise acquiring knowledge of or detecting one or more mutations in an SPOP gene and/or a TMPRSS2-ERG fusion nucleic acid comprising a fusion between a TMPRSS2 gene, or a portion thereof, and a ERG gene, or a portion thereof, in a sample from the individual having a mHSPC or a nmCRPC. In some embodiments, the presence of the one or more mutations in the SPOP gene and/or the TMPRSS2-ERG fusion nucleic acid in the sample indicate that the individual has a higher likelihood of benefiting from a treatment comprising a hormone therapy (e.g., a novel hormonal therapy).

### A. Individuals

Certain aspects of the disclosure provide for acquiring knowledge of or detecting a biomarker in a sample from an individual having a mHSPC or a nmCRPC. In some embodiments, the mHSPC is a de novo mHSPC. In other embodiments, the mHSPC is a recurrent HSPC. In some embodiment, the

In some instances, the individual has a cancer or is at risk of having a mHSPC or a nmCRPC. For example, in some instances, the individual has a genetic predisposition to a mHSPC or a nmCRPC (e.g., having a genetic mutation that increases their baseline risk for developing a mHSPC or a nmCRPC). In some instances, the individual has been exposed to an environmental perturbation (e.g., radiation or a chemical) that increases their risk for developing a mHSPC or a nmCRPC. In some instances, the subject is in need of being monitored for development of a mHSPC or a nmCRPC. In some instances, the subject is in need of being monitored for cancer progression or regression, e.g., after being treated with an anti-cancer therapy (or anti-cancer treatment). In some instances, the subject is in need of being monitored for relapse of a mHSPC or a nmCRPC. In some instances, the subject is in need of being monitored for minimum residual disease (MRD). In some instances, the subject has been, or is being treated, with an anti-cancer therapy. In some instances, the subject has not been treated with an anti-cancer therapy (or anti-cancer treatment).

In some embodiments, the subject is a human. In some embodiments, the subject is a non-human mammal.

### B. Biomarkers

Certain aspects of the methods provided herein relate to acquiring knowledge of or detecting one or more mutations in the SPOP gene and/or the TMPRSS2-ERG fusion nucleic acid comprising a fusion between the TMPRSS2 gene, or the portion thereof, and the ERG gene, or the portion thereof, in a sample from an individual, e.g., having a mHSPC or a nmCRPC.

### (i) SPOP

Certain aspects of the present disclosure relate to one or more mutations in an SPOP gene.

As used herein “SPOP” refers to a gene encoding an SPOP mRNA or polypeptide. The SPOP gene encodes the speckle type BTB/POZ protein. SPOP is also known as TEF2, BTBD32, NSDVS1, NSDVS2, NEDMACE, NEDMIDF. In some embodiments, an SPOP gene is a human SPOP gene. An exemplary SPOP gene is represented by NCBI Gene ID No. 8405. Exemplary SPOP transcript sequences are represented by NCBI Ref. Seq. NM_001007226.1. Exemplary amino acid sequences of an SPOP polypeptide are represented by NCBI Ref. Seq. NP_001007227.1. I

In some embodiments, the one or more mutations in the SPOP gene comprise one or more of a substitution of one or more nucleotides, an insertion of one or more nucleotides, or a deletion of one or more nucleotides. In some embodiments, the one or more mutations in the SPOP gene comprise one or more of a genomic rearrangement, an alteration in a promoter, a gene fusion, or a copy number alteration. In some embodiments, the one or more mutations in the SPOP gene comprise a gene copy number alteration. In some embodiments, the one or more mutations comprise a gene amplification. In some embodiments, the one or more mutations in the SPOP gene comprise a gene deletion, e.g., a deletion of the entire SPOP gene or of a portion of the SPOP gene. In some embodiments, the one or more mutations in the SPOP gene comprise a point mutation. In some embodiments, the one or more mutations in the SPOP gene comprise a single nucleotide polymorphism. In some embodiments, the one or more mutations comprise one or more mutations in an exon and/or an intron of the gene. In some embodiments, the one or more mutations in the SPOP gene comprise a non-synonymous mutation. In some embodiments, the one or more mutations in the SPOP gene comprise a missense mutation. In some embodiments, the one or more mutations in the SPOP gene comprise a nonsense mutation. In some embodiments, the one or more mutations in the SPOP gene comprise a gain-of-function mutation, e.g., an activating mutation. In some embodiments, the one or more mutations in the SPOP gene comprise a loss-of-function mutation, e.g., an inactivating mutation. In some embodiments, the one or more mutations in the SPOP gene result in a frameshift. In some embodiments, the one or more mutations in the SPOP gene result in a premature stop codon. In some embodiments, the one or more mutations in the SPOP gene comprise a functional alteration. In some embodiments, the one or more mutations in the SPOP gene comprise a mutation that alters the function of the SPOP polypeptide by the mutated SPOP gene. In some embodiments, the one or more mutations in the SPOP gene comprise a complex insertion. In some embodiments, the one or more mutations in the SPOP gene comprise a complex deletion. In some embodiments, the one or more mutations in the SPOP gene comprise a mutation in a splice site. In some embodiments, the one or more mutations in the SPOP gene alter the splicing of an SPOP mRNA molecule encoded by the SPOP gene. In some embodiments, the one or more mutations in the SPOP gene comprise an insertion of one or more nucleotides. In some embodiments, the insertion comprises an insertion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the insertion comprises an insertion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the insertion comprises an insertion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations in the SPOP gene comprise a deletion of one or more nucleotides. In some embodiments, the deletion comprises a deletion of between about 1 and about 5 nucleotides, between about 5 and about 10 nucleotides, between about 10 and about 20 nucleotides, between about 20 and about 30 nucleotides, between about 30 and about 40 nucleotides, or between about 40 and about 50 nucleotides. In some embodiments, the deletion comprises a deletion of between about 50 and about 100 nucleotides, between about 100 and about 200 nucleotides, between about 200 and about 300 nucleotides, between about 300 and 400 nucleotides, between about 400 and about 500 nucleotides, between about 500 and about 600 nucleotides, between about 600 and about 700 nucleotides, between about 700 and about 800 nucleotides, between about 800 and about 900 nucleotides, or between about 900 and about 1000 nucleotides. In some embodiments, the deletion comprises a deletion of between about 1000 and about 1500 nucleotides, between about 1500 and about 2000 nucleotides, between about 2000 and about 2500 nucleotides, between about 2500 and about 3000 nucleotides, between about 3000 and about 3500 nucleotides, between about 3500 and about 4000 nucleotides, between about 4000 and about 4500 nucleotides, between about 4500 and about 5000 nucleotides, between about 5000 and about 5500 nucleotides, between about 5500 and about 6000 nucleotides, between about 6000 and about 6500 nucleotides, between about 6500 and about 7000 nucleotides, between about 7000 and about 7500 nucleotides, between about 7500 and about 8000 nucleotides, between about 8000 and about 8500 nucleotides, between about 8500 and about 9000 nucleotides, between about 9000 and about 9500 nucleotides, or between about 9500 and about 10000 nucleotides. In some embodiments, the one or more mutations in the SPOP gene result in a substitution, insertion, or deletion of one or more amino acid residues in an SPOP polypeptide encoded by the mutated SPOP gene. In some embodiments, the one or more mutations in the SPOP gene result in a substitution of one or more amino acid residues in an SPOP polypeptide encoded by the mutated SPOP gene. In some embodiments, the one or more mutations in the SPOP gene result in a deletion of one or more amino acid residues in an SPOP polypeptide encoded by the mutated SPOP gene. In some embodiments, the one or more mutations in the SPOP gene result in an insertion of one or more amino acid residues in an SPOP polypeptide encoded by the mutated SPOP gene.

In some embodiments, the one or more mutations in the SPOP gene comprise one or more of a 305T>G mutation, a 397T>G mutation, a 399C>G mutation, a 393G>C mutation, a 304T>G mutation, a 374T>G mutation, a 398T>C mutation, a 398T>G mutation, a 259T>A mutation, a 260A>G mutation, a 304T>A mutation, a 356G>A mutation, a 375T>G mutation, a 389A>T mutation, a 391T>C mutation, a 391T>G mutation, a 393G>T mutation, a 397T>A mutation, a 399C>A mutation, or any combination thereof. In some embodiments, the one or more mutations in the SPOP gene result in decreased activity of an SPOP polypeptide encoded by the mutated SPOP gene. In some embodiments, the one or more mutations in the SPOP gene result in one or more amino acid substitutions in an SPOP polypeptide encoded by the mutated SPOP gene. In some embodiments, the mutated SPOP gene encodes an SPOP polypeptide comprising one or more amino acid substitutions corresponding to one or more of R45W, R70*, Y87C, Y87N, Y87S, Y87F, Y87D, F102C, F102V, F102S, F102I, F102L, F104V, F102Y, F104C, F104S, F104I, A116G, M117V, S119N, F125I, F125V, F125L, F125C, K129E, D130V, D130N, W131G, W131C, W131R, W131L, W131S, F133L, F133V, F133I, F133C, F133S, K134N, A277V, and L282R, or any combination thereof. In some embodiments, the SPOP polypeptide encoded by the mutated SPOP gene has decreased activity as compared to an SPOP polypeptide encoded by an SPOP gene that does not comprise the one or more mutations. In some embodiments, the SPOP polypeptide encoded by the mutated SPOP gene is oncogenic. In some embodiments, the SPOP polypeptide encoded by the mutated SPOP gene promotes cancer cell survival, angiogenesis, cancer cell proliferation, genomic instability, or any combination thereof.

### (ii) TMPRSS2-ERG Fusion Nucleic Acid

Certain aspects of the present disclosure relate to a TMPRSS2-ERG fusion nucleic acid. A TMPRSS2-ERG fusion nucleic acid of the present disclosure may relate to any chromosomal translocation, fusion, or rearrangement involving the locus of a TMPRSS2 gene and an ERG gene. In some embodiments, the TMPRSS2-ERG fusion nucleic acid comprises a fusion between the TMPRSS2 gene, or the portion thereof, and the ERG gene, or the portion thereof.

As used herein “TMPRSS2” refers to a gene encoding a TMPRSS2 mRNA or polypeptide. The TMPRSS2 gene encodes the transmembrane serine protease 2. TMPRSS2 is also known as PRSS10. In some embodiments, a TMPRSS2 gene is a human TMPRSS2 gene. An exemplary TMPRSS2 gene is represented by NCBI Gene ID No. 7113. Exemplary TMPRSS2 transcript sequences are represented by NCBI Ref. Seq. NM_005656.4. Exemplary amino acid sequences of a TMPRSS2 polypeptide are represented by NCBI Ref. Seq. NP_005647.3.

As used herein “ERG” refers to a gene encoding an ERG mRNA or polypeptide. The ERG gene encodes the ETS transcription factor. ERG is also known as P55 and ERG-3. In some embodiments, an ERG gene is a human ERG gene. An exemplary ERG gene is represented by NCBI Gene ID No. 2078. Exemplary ERG transcript sequences are represented by NCBI Ref. Seq. NM_004449.4. Exemplary amino acid sequences of an ERG polypeptide are represented by NCBI Ref. Seq. NP_004440.1.

In some embodiments, the TMPRSS2-ERG fusion nucleic acid molecule of the disclosure comprises, in the 5′ to 3′ direction, a TMPRSS2 gene, or a portion thereof fused to an ERG gene, or a portion thereof. In some embodiments, the TMPRSS2-ERG fusion nucleic acid molecule of the disclosure comprises or results from a breakpoint within an intron of TMPRSS2 and/or an intron of ERG. In some embodiments, the TMPRSS2-ERG fusion nucleic acid molecule of the disclosure comprises or results from a breakpoint within an exon of TMPRSS2 and/or an exon of ERG. In some embodiments, the TMPRSS2-ERG fusion nucleic acid encodes a TMPRSS2-ERG fusion polypeptide. In some embodiments, the TMPRSS2-ERG fusion nucleic acid encoded by the TMPRSS2-ERG fusion nucleic acid is oncogenic. In some embodiments, the TMPRSS2-ERG fusion nucleic acid encoded by the TMPRSS2-ERG fusion nucleic acid promotes cancer cell survival, angiogenesis, cancer cell proliferation, genomic instability, or any combination thereof.

### C. Detection of Gene Alterations in Nucleic Acids

In some embodiments, the methods provided herein comprise detecting one or more mutations in an SPOP gene and/or a TMPRSS2-ERG fusion nucleic acid comprising a fusion between a TMPRSS2 gene, or a portion thereof, and an ERG gene, or a portion thereof.

In some embodiments, the one or more mutations in the SPOP gene and/or the TMPRSS2-ERG fusion nucleic acid comprising a fusion between the TMPRSS2 gene, or the portion thereof, and the ERG gene, or the portion thereof, are detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

### (i) In Situ Hybridization Methods

In some embodiments, the one or more mutations in the SPOP gene and/or the TMPRSS2-ERG fusion nucleic acid comprising a fusion between the TMPRSS2 gene, or the portion thereof, and the ERG gene, or the portion thereof, are detected using an in situ hybridization method, such as a fluorescence in situ hybridization (FISH) method.

In some embodiments, FISH analysis is used to identify the chromosomal rearrangement resulting in the mutations as described herein. In some embodiments, FISH analysis is used to identify an RNA molecule comprising the one or more mutations in the SPOP gene and/or the TMPRSS2-ERG fusion nucleic acid. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH.

### (ii) Array-Based Methods

In some embodiments, the one or more mutations in the SPOP gene and/or the TMPRSS2-ERG fusion nucleic acid comprising a fusion between the TMPRSS2 gene, or the portion thereof, and the ERG gene, or the portion thereof, are detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

(iii) Amplification-Based Methods

In some embodiments, the one or more mutations in the SPOP gene and/or the TMPRSS2-ERG fusion nucleic acid comprising a fusion between the TMPRSS2 gene, or the portion thereof, and the ERG gene, or the portion thereof, are detected using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of the one or more mutations in the SPOP gene and/or the TMPRSS2-ERG fusion nucleic acid) in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

### (iv) Sequencing

In some embodiments, the one or more mutations in the SPOP gene and/or the TMPRSS2-ERG fusion nucleic acid comprising a fusion between the TMPRSS2 gene, or the portion thereof, and the ERG gene, or the portion thereof, are detected by sequencing.

In some embodiments, sequencing comprises providing a plurality of nucleic acid molecules obtained from the sample; amplifying nucleic acid molecules from the plurality of nucleic acid molecules; capturing nucleic acid molecules from the amplified nucleic acid molecules; and sequencing, by a sequencer, the captured nucleic acid molecules to obtain a plurality of sequence reads corresponding to one or more genomic loci within a subgenomic interval in the sample. In some embodiments, the plurality of nucleic acid molecules comprises a mixture of tumor nucleic acid molecules and non-tumor nucleic acid molecules.

In some embodiments, amplification of the nucleic acid molecules is performed by a polymerase chain reaction (PCR) technique, a non-PCR amplification technique, or an isothermal amplification technique.

In some embodiments, sequencing further comprises ligating one or more adapters onto one or more nucleic acid molecules from the plurality of nucleic acid molecules. In some embodiments, the adapters comprise one or more of amplification primer sequences, flow cell adapter hybridization sequences, unique molecular identifier sequences, substrate adapter sequences, or sample index sequences.

In some embodiments, nucleic acid molecules from a library are isolated, e.g., using solution hybridization, thereby providing a library catch. The library catch, or a subgroup thereof, can be sequenced. Accordingly, the methods described herein can further include analyzing the library catch. In some embodiments, the library catch is analyzed by a sequencing method, e.g., a next-generation sequencing method as described herein. In some embodiments, the method includes isolating a library catch by solution hybridization, and subjecting the library catch to nucleic acid sequencing. In certain embodiments, the library catch is re-sequenced.

In some embodiments, the captured nucleic acid molecules are captured from the amplified nucleic acid molecules by hybridization to one or more bait molecules. In some embodiments, the one or more bait molecules comprise one or more nucleic acid molecules, each comprising a region that is complementary to a region of a captured nucleic acid molecule. In some embodiments, the one or more bait molecules each comprise a capture moiety. In some embodiments, the capture moiety is biotin.

Any method of sequencing known in the art can be used. Sequencing of nucleic acids, e.g., isolated by solution hybridization, are typically carried out using next-generation sequencing (NGS). Sequencing methods suitable for use herein are described in the art, e.g., as described in International Patent Application Publication No. WO 2012/092426. In some embodiments, sequencing is performed using a massively parallel sequencing (MPS) technique, whole genome sequencing (WGS), whole exome sequencing, targeted sequencing, direct sequencing, next-generation sequencing (NGS), or a Sanger sequencing technique.

In some embodiments, sequencing comprises detecting alterations present in the genome, whole exome or transcriptome of an individual. In some embodiments, sequencing comprises DNA and/or RNA sequencing, e.g., targeted DNA and/or RNA sequencing. In some embodiments, the sequencing comprises detection of a change (e.g., an increase or decrease) in the level of a gene or gene product, e.g., a change in expression of a gene or gene product described herein.

Sequencing can, optionally, include a step of enriching a sample for a target RNA. In other embodiments, sequencing includes a step of depleting the sample of certain high abundance RNAs, e.g., ribosomal or globin RNAs. The RNA sequencing methods can be used, alone or in combination with the DNA sequencing methods described herein. In one embodiment, sequencing includes a DNA sequencing step and an RNA sequencing step. The methods can be performed in any order. For example, the method can include confirming by RNA sequencing the expression of an alteration described herein, e.g., confirming expression of a mutation or a fusion detected by the DNA sequencing methods of the invention. In other embodiments, sequencing

### (v) Nucleic Acid Detection Reagents

In some aspects, provided herein are reagents for detecting the one or more mutations in the SPOP gene and/or the TMPRSS2-ERG fusion nucleic acid comprising a fusion between the TMPRSS2 gene, or the portion thereof, and the ERG gene, or the portion thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid.

In some embodiments, the detection reagents comprise a bait. In some embodiments, the bait comprises a capture nucleic acid molecule configured to hybridize to a target nucleic acid molecule comprising the one or more mutations in the SPOP gene and/or the TMPRSS2-ERG fusion nucleic acid. In some embodiments, the bait comprises a capture nucleic acid molecule configured to hybridize to a target nucleic acid molecule, e.g., a target nucleic acid molecule comprising nucleotide sequences of one or more genes selected from an SPOP gene, a TMPRSS2 gene, or an ERG gene. In some embodiments, the capture nucleic acid molecule is configured to hybridize to a nucleic acid molecule encoding one or more genes selected from an SPOP gene, a TMPRSS2 gene, or an ERG gene. In some embodiments, the bait comprises a capture nucleic acid molecule configured to hybridize to a target nucleic acid molecule. In some embodiments, the capture nucleic acid molecule is configured to hybridize to a nucleic acid molecule encoding one or more of a SPOP gene, a TMPRSS2 gene, or an ERG gene. In some embodiments, the capture nucleic acid molecule is configured to hybridize to a fragment of a nucleic acid molecule comprising the one or more mutations in the SPOP gene and/or the TMPRSS2-ERG fusion nucleic acid. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises a nucleic acid sequence configured to hybridize to a nucleic acid molecule comprising the one or more mutations in the SPOP gene and/or the TMPRSS2-ERG fusion nucleic acid, e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides.

In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule is between about 5 and about 25 nucleotides in length, between about 5 and about 300 nucleotides in length, between about 100 and about 300 nucleotides in length, between about 130 and about 230 nucleotides in length, or between about 150 and about 200 nucleotides in length.

In some embodiments, the capture nucleic acid is configured to hybridize to a breakpoint in the TMPRSS2-ERG fusion nucleic acid, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint. In some embodiments, the capture nucleic acid is configured to hybridize to a nucleotide sequence in an intron or an exon of the TMPRSS2 gene in the fusion nucleic acid and/or to a nucleotide sequence in an intron or an exon of an ERG gene in the fusion nucleic acid, and/or to a nucleotide sequence in a breakpoint the fusion (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid is configured to hybridize to a breakpoint joining an intron or a exon of a TMPRSS2 gene and an intron or an exon of an ERG gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, the bait hybridizes to a nucleotide sequence comprising the one or more mutations in the SPOP gene and/or the TMPRSS2-ERG fusion nucleic acid, and a sequence on either side of the alteration (e.g., any of 1, 2, 3, 4, 5, 6, 7, 8, 9, or 10 nucleotides on either side of the alteration, or any of between about 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the alteration).

The baits described herein can be used for selection of exons and short target sequences.

In some embodiments, a bait of the disclosure distinguishes a nucleic acid, e.g., a genomic or transcribed nucleic acid, e.g., a cDNA or RNA, comprising the one or more mutations in the SPOP gene and/or the TMPRSS2-ERG fusion nucleic acid, from a reference nucleotide sequence, e.g., a nucleotide sequence not having the one or more gene alterations.

In some embodiments, the detection reagents comprise a probe. In some embodiments, a probe provided herein comprises a nucleic acid sequence configured to hybridize to a target nucleic acid comprising the one or more mutations in the SPOP gene and/or the TMPRSS2-ERG fusion nucleic acid, or a fragment or portion thereof. In some embodiments, a probe provided herein comprises a nucleic acid sequence configured to hybridize to a target nucleic acid molecule, e.g., a target nucleic acid molecule comprising nucleotide sequences of an SPOP gene, a TMPRSS2 gene, or an ERG gene In some embodiments, the probe comprises a nucleic acid sequence configured to hybridize to a fragment or portion of a nucleic acid molecule encoding An SPOP gene, a TMPRSS2 gene, or an ERG gene. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides. In some embodiments, the probe comprises a nucleic acid sequence configured to hybridize to a nucleic acid molecule encoding the one or more mutations in the SPOP gene and/or the TMPRSS2-ERG fusion nucleic acid plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides.

In some embodiments, the probe comprises a nucleotide sequence configured to hybridize to a breakpoint in the TMPRSS2-ERG fusion nucleic acid, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint. In some embodiments, the probe is configured to hybridize to a nucleotide sequence in an intron or an exon of the TMPRSS2 gene in the fusion nucleic acid and/or to a nucleotide sequence in an intron or an exon of an ERG gene in the fusion nucleic acid, and/or to a nucleotide sequence in a breakpoint the fusion (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe is configured to hybridize to a breakpoint joining an intron or a exon of a TMPRSS2 gene and an intron or an exon of an ERG gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of the one or more mutations in the SPOP gene and/or the TMPRSS2-ERG fusion nucleic acid. For example, a probe provided herein may be used for detecting the one or more mutations in the SPOP gene and/or the TMPRSS2-ERG fusion nucleic acid in a sample, e.g., a sample obtained from an individual. In some embodiments, the probe may be used for identifying cells or tissues that express the one or more mutations in the SPOP gene and/or the TMPRSS2-ERG fusion nucleic acid, e.g., by measuring levels of a nucleic acid molecule (e.g., a transcribed nucleic acid molecule, such as an RNA or an mRNA) comprising the one or more alterations. In some embodiments, the probe may be used for detecting levels of the one or more mutations in the SPOP gene and/or the TMPRSS2-ERG fusion nucleic acid, e.g., mRNA levels, in a sample of cells from an individual.

In some embodiments, a probe of the disclosure distinguishes a nucleic acid, e.g., a genomic or transcribed nucleic acid, e.g., a cDNA or RNA, comprising the one or more mutations in the SPOP gene and/or the TMPRSS2-ERG fusion nucleic acid, from a reference nucleotide sequence, e.g., a nucleotide sequence not comprising the one or more mutations in the SPOP gene and/or the TMPRSS2-ERG fusion nucleic acid.

Also provided herein are isolated pairs of allele-specific probes, wherein, for example, the first probe of the pair specifically hybridizes to a nucleic acid molecule comprising the one or more mutations in the SPOP gene and/or the TMPRSS2-ERG fusion nucleic acid, and the second probe of the pair specifically hybridizes to a corresponding wild type sequence (e.g., a wild type SPOP, TMPRSS2, or ERG gene nucleotide sequence). Probe pairs can be designed and produced for any of the gene alterations described herein and are useful in detecting a somatic mutation in a sample. In some embodiments, a first probe of a pair specifically hybridizes to the one or more mutations in the SPOP gene and/or the TMPRSS2-ERG fusion nucleic acid, and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10⁵ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising the one or more mutations in the SPOP gene and/or the TMPRSS2-ERG fusion nucleic acid.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results the one or more mutations in the SPOP gene and/or the TMPRSS2-ERG fusion nucleic acid. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a nucleotide sequence encoding the one or more mutations in the SPOP gene and/or the TMPRSS2-ERG fusion nucleic acid, and a sequence on either side of the one or more gene alterations (e.g., any of 1, 2, 3, 4, 5, 6, 7, 8, 9, or 10 nucleotides on either side of the one or more gene alterations, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the one or more gene alterations). In some embodiments, the detection reagents comprise an oligonucleotide, e.g., useful as a primer. In some embodiments, an oligonucleotide, e.g., a primer, provided herein comprises a nucleotide sequence configured to hybridize to a target nucleic acid molecule comprising the one or more mutations in the SPOP gene and/or the TMPRSS2-ERG fusion nucleic acid, or a fragment or portion thereof. In some embodiments, an oligonucleotide, e.g., a primer, provided herein is configured to hybridize to a nucleic acid molecule encoding an SPOP gene, a TMPRSS2 gene, or an ERG gene. In some embodiments, the oligonucleotide comprises a nucleic acid sequence configured to hybridize to a nucleotide sequence encoding the one or more mutations in the SPOP gene and/or the TMPRSS2-ERG fusion nucleic acid, e.g., plus or minus any of between about 10 and about 12, about 12 and about 15, about 15 and about 17, about 17 and about 20, about 20 and about 25, or about 25 and about 30, or more nucleotides.

In some embodiments, the gene alteration is a gene fusion or a rearrangement. In some embodiments, the oligonucleotide comprises a nucleotide sequence configured to hybridize to a breakpoint of a gene fusion or rearrangement, and may be further configured to hybridize to between about 10 and about 12, about 12 and about 15, about 15 and about 17, about 17 and about 20, about 20 and about 25, or about 25 and about 30, or more nucleotides flanking either side of the breakpoint. In some embodiments, the oligonucleotide is configured to hybridize to a nucleotide sequence in an intron or an exon of a first gene of a gene fusion or rearrangement, and/or to a nucleotide sequence in an intron or an exon of a second gene of a gene fusion or rearrangement, and/or to a nucleotide sequence in a breakpoint of a gene fusion or rearrangement (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide is configured to hybridize to a breakpoint joining an intron or a exon of first gene and an intron or an exon of a second gene (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide comprises a nucleotide sequence corresponding to a nucleic acid molecule an SPOP gene, a TMPRSS2 gene, or an ERG gene. In some embodiments, the oligonucleotide comprises a nucleotide sequence corresponding to a nucleic acid molecule encoding the one or more mutations in the SPOP gene and/or the TMPRSS2-ERG fusion nucleic acid. In some embodiments, the oligonucleotide comprises a nucleotide sequence corresponding to a fragment or a portion of a nucleic acid molecule encoding an SPOP gene, a TMPRSS2 gene, or an ERG gene. In some embodiments, the oligonucleotide comprises a nucleotide sequence corresponding to a fragment or a portion of a nucleic acid molecule the one or more mutations in the SPOP gene and/or the TMPRSS2-ERG fusion nucleic acid. In some embodiments, the fragment or portion comprises between about 10 and about 30 nucleotides, between about 12 and about 20 nucleotides, or between about 12 and about 17 nucleotides.

In some embodiments, the oligonucleotide comprises a nucleotide sequence complementary to a nucleic acid molecule encoding an SPOP gene, a TMPRSS2 gene, or an ERG gene. In some embodiments, the oligonucleotide comprises a nucleotide sequence complementary to a nucleic acid molecule the one or more mutations in the SPOP gene and/or the TMPRSS2-ERG fusion nucleic acid. In some embodiments, the oligonucleotide comprises a nucleotide sequence complementary to a fragment or a portion of a nucleic acid molecule encoding an SPOP gene, a TMPRSS2 gene, or an ERG gene. In some embodiments, the oligonucleotide comprises a nucleotide sequence complementary to a fragment or a portion of a nucleic acid molecule encoding the one or more mutations in the SPOP gene and/or the TMPRSS2-ERG fusion nucleic acid In some embodiments, the fragment or portion comprises between about 10 and about 30 nucleotides, between about 12 and about 20 nucleotides, or between about 12 and about 17 nucleotides. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule comprising the one or more mutations in the SPOP gene and/or the TMPRSS2-ERG fusion nucleic acid, or a fragment thereof, e.g., using PCR. In some embodiments, the oligonucleotide may be used to sequence a nucleic acid molecule comprising the one or more mutations in the SPOP gene and/or the TMPRSS2-ERG fusion nucleic acid, or a fragment thereof. In some embodiments, the oligonucleotide may be used to amplify a nucleic acid molecule comprising a breakpoint of a fusion or rearrangement provided herein, e.g., using PCR. In some embodiments, the oligonucleotide may be used to sequence a nucleic acid molecule comprising a breakpoint a fusion or rearrangement provided herein.

In some embodiments, pairs of oligonucleotides, e.g., pairs of primers, are provided herein, which are configured to hybridize to a nucleic acid molecule comprising the one or more mutations in the SPOP gene and/or the TMPRSS2-ERG fusion nucleic acid, or a fragment thereof. In some embodiments, a pair of oligonucleotides of the disclosure may be used for directing amplification of a nucleic acid molecule or fragment thereof comprising the one or more mutations in the SPOP gene and/or the TMPRSS2-ERG fusion nucleic acid, e.g., using a PCR reaction. In some embodiments, pairs of oligonucleotides, e.g., pairs of primers, are provided herein, which are configured to hybridize to a nucleic acid molecule encoding the one or more mutations in the SPOP gene and/or the TMPRSS2-ERG fusion nucleic acid, e.g., for use in directing amplification, e.g., using a PCR reaction. In some embodiments, pairs of oligonucleotides, e.g., pairs of primers, are provided herein, which are configured to hybridize to a nucleic acid molecule comprising a breakpoint of a fusion or rearrangement provided herein, e.g., for use in directing amplification of a fusion nucleic acid molecule, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a nucleic acid molecule comprising the one or more mutations in the SPOP gene and/or the TMPRSS2-ERG fusion nucleic acid, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, comprising the one or more mutations in the SPOP gene and/or the TMPRSS2-ERG fusion nucleic acid, from a reference nucleotide sequence, e.g., a nucleotide sequence not comprising the one or more mutations in the SPOP gene and/or the TMPRSS2-ERG fusion nucleic acid.

In some embodiments, the oligonucleotide, e.g., the primer, hybridizes to the one or more mutations in the SPOP gene and/or the TMPRSS2-ERG fusion nucleic acid, and a sequence on either side of the one or more mutations in the SPOP gene and/or the TMPRSS2-ERG fusion nucleic acid, e.g., any of 1, 2, 3, 4, 5, 6, 7, 8, 9, or 10 nucleotides on either side of the one or more gene alterations, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the one or more gene alterations).

### D. Detection of Gene Alterations in Polypeptides

Also provided herein are methods comprising detecting one or more mutations in an SPOP gene and/or a TMPRSS2-ERG fusion nucleic acid comprising a fusion between a TMPRSS2 gene, or a portion thereof, and an ERG gene, or a portion thereof in an encoded polypeptide, or a fragment thereof. An SPOP polypeptide encoded by the mutated SPOP gene and/or a TMPRSS2-ERG fusion polypeptide encoded by the TMPRSS2-ERG fusion nucleic acid, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, an SPOP polypeptide encoded by the mutated SPOP gene and/or a TMPRSS2-ERG fusion polypeptide encoded by the TMPRSS2-ERG fusion nucleic acid, or a fragment thereof, can be distinguished from a reference polypeptide, e.g., a corresponding non-mutant or wild type protein or polypeptide, with an antibody or antibody fragment that reacts differentially with a mutant protein or polypeptide (e.g., a polypeptide encoded by a gene comprising one or more gene alterations of the disclosure) as compared to a reference protein or polypeptide. In some embodiments, an SPOP polypeptide encoded by the mutated SPOP gene and/or a TMPRSS2-ERG fusion polypeptide encoded by the TMPRSS2-ERG fusion nucleic acid, or a fragment thereof, can be distinguished from a reference polypeptide, e.g., a corresponding non-mutant or wild type protein or polypeptide, by reaction with a detection reagent, e.g., a substrate, e.g., a substrate for catalytic activity, e.g., phosphorylation (e.g., kinase activity), dephosphorylation (e.g., phosphatase activity), or another suitable catalytic activity.

In some aspects, methods of detection of an SPOP polypeptide encoded by an SPOP gene comprising one or more mutations, and/or a TMPRSS2-ERG fusion polypeptide encoded by a TMPRSS2-ERG fusion nucleic acid, or a fragment thereof, are provided, comprising contacting a sample, e.g., a sample described herein, comprising an SPOP polypeptide encoded by the mutated SPOP gene and/or a TMPRSS2-ERG fusion polypeptide encoded by the TMPRSS2-ERG fusion nucleic acid with a detection reagent (e.g., an antibody), and determining if an SPOP polypeptide encoded by the mutated SPOP gene and/or a TMPRSS2-ERG fusion polypeptide encoded by the TMPRSS2-ERG fusion nucleic acid is present.

### E. Samples

A variety of materials (such as tissues) can be the source of protein samples used in the methods provided herein. For example, the source of the sample can be a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

The disclosed methods and systems may be used with any of a variety of samples (also referred to herein as specimens) comprising nucleic acids (e.g., DNA or RNA), and/or polypeptides, that are collected from a subject. Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain embodiments, the sample may be frozen sample or a formalin-fixed paraffin-embedded (FFPE) sample.

In some embodiments, the sample may be collected by tissue resection (e.g., surgical resection), needle biopsy, skin biopsy, endoscopic biopsy, fine needle aspiration, or a cytology smear, scrapings, washings or lavages, etc.

In some embodiments, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise mRNA, DNA, cell-free DNA (cfDNA), circulating tumor DNA (ctDNA), cell-free RNA from a cancer, or any combination thereof.

In some embodiments, the sample may comprise one or more premalignant or malignant cells. Premalignant, as used herein, refers to a cell or tissue that is not yet malignant but is poised to become malignant. In certain embodiments, the sample may be acquired from a solid tumor, a soft tissue tumor, or a metastatic lesion. In certain instances, the sample may be acquired from a hematologic malignancy or pre-malignancy. In other embodiments, the sample may comprise a tissue or cells from a surgical margin. In certain instances, the sample may comprise tumor-infiltrating lymphocytes. In some instances, the sample may comprise one or more non-malignant cells. In some embodiments, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some embodiments, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some embodiments, the sample may comprise any normal control (e.g., a normal adjacent tissue (NAT)) if no primary control is available. In some embodiments, the sample may be or may comprise histologically normal tissue. In some embodiments, the method includes evaluating a sample, e.g., a histologically normal sample (e.g., from a surgical tissue margin) using the methods described herein. In some embodiments, the disclosed methods may further comprise acquiring a sub-sample enriched for non-tumor cells, e.g., by macro-dissecting non-tumor tissue from said NAT in a sample not accompanied by a primary control. In some embodiments, the disclosed methods may further comprise determining that no primary control and no NAT is available, and marking said sample for analysis without a matched control.

In some embodiments, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some embodiments, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some embodiments, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some embodiments, DNA is extracted from nucleated cells from the sample. In some embodiments, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some embodiments, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly(A)-tailed mRNA fraction of the total RNA, ribosomal RNA (rRNA) or fragments thereof, transfer RNA (tRNA) or fragments thereof, and mitochondrial RNA or fragments thereof. In some embodiments, RNA may be extracted from the sample and converted to complementary DNA (cDNA) using, e.g., a reverse transcription reaction. In some embodiments, the cDNA is produced by random-primed cDNA synthesis methods. In other embodiments, the cDNA synthesis is initiated at the poly(A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some embodiments, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some embodiments, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some embodiments, as noted above, the sample comprises nucleic acid (e.g., DNA, RNA (or a cDNA derived from the RNA), or both), e.g., from a tumor or from normal tissue. In certain embodiments, the sample may further comprise a non-nucleic acid component, e.g., cells, protein, carbohydrate, or lipid, e.g., from the tumor or normal tissue.

### F. Anti-Cancer Treatments

Certain aspects of the present disclosure relate to anti-cancer treatments. In some embodiments, the anti-cancer treatment is a hormonal therapy (e.g., a novel hormonal therapy). In some embodiments, the treatment does not comprise a chemotherapy (e.g., a taxane). In some embodiments, the treatment further comprises an additional anti-cancer therapy.

Any of the anti-cancer therapies (optionally as monotherapies or in combination with another therapy or treatment) may find use in any of the methods described herein.

### (i) Hormone Therapies

Certain aspects of the present disclosure relate to hormonal therapies.

In some embodiments, the hormone therapy is a novel hormonal therapy (NHT). In some embodiments, the NHT comprises a second generation hormonal therapy. In some embodiments, the NHT comprises one or more of abiraterone, enzalutamide, apalutamide, and darolutamide, or any combination thereof.

In some embodiments, the treatment further comprises an anti-androgen agent. In some embodiments, the anti-androgen agent is bicalutamide, flutamide, nilutamide, enzalutamide, apalutamide, or darolutamide.

In some embodiments, the treatment further comprises an androgen deprivation therapy (ADT). In some embodiments, the ADT comprises In some embodiments, the ADT comprises one or more of a orchiectomy, a LHRH agonist, a LHRH antagonist, an anti-androgen agent, an estrogen, and an androgen synthesis inhibitor, or any combination thereof.

In some embodiments, wherein the orchiectomy is a bilateral orchiectomy. In some embodiments, wherein the LHRH agonist is goserelin acetate, histrelin acetate, leuprolide acetate, or triptorelin acetate. In some embodiments, the anti-androgen agent is bicalutamide, flutamide, nilutamide, enzalutamide, apalutamide, or darolutamide. In some embodiments, the estrogen is diethylstilbestrol. In some embodiments, the androgen synthesis inhibitor is abiraterone or ketoconazole

### (ii) Chemotherapies

Certain aspects of the present disclosure relate to chemotherapies.

In some embodiments, the methods provided herein comprise identifying a treatment for an individual, or treating an individual, wherein the treatment does not comprise a chemotherapy (e.g., a taxane). Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredepa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above.

Some non-limiting examples of chemotherapeutic drugs are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

(iii) Anti-Cancer Therapies

Certain aspects of the disclosure provide for anti-cancer therapies.

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-7, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v 33 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-7 inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TGO2 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient.

In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

### III. Kits

Also provided herein are kits for detecting one or more mutations in an SPOP gene and/or a TMPRSS2-ERG fusion nucleic acid comprising a fusion between a TMPRSS2 gene, or a portion thereof, and an ERG gene, or a portion thereof, in a sample from an individual having a cancer, e.g., a mHSPC or a nmCRPC. In some embodiments, a kit provided herein comprises a reagent (e.g., one or more oligonucleotides, primers, probes or baits of the present disclosure) for detecting one or more mutations in an SPOP gene and/or a TMPRSS2-ERG fusion nucleic acid comprising a fusion between a TMPRSS2 gene, or a portion thereof, and an ERG gene, or a portion thereof. In some embodiments, the kit comprises a reagent (e.g., one or more oligonucleotides, primers, probes or baits of the present disclosure) for detecting a wild-type counterpart of the SPOP, TMPRSS2 or ERG genes. In some embodiments, the reagent comprises one or more oligonucleotides, primers, probes or baits of the present disclosure capable of hybridizing to a nucleic acid molecule comprising or encoding one or more mutations in an SPOP gene, or to a wild-type counterpart. In some embodiments, the reagent comprises one or more oligonucleotides, primers, probes or baits of the present disclosure capable of hybridizing to a TMPRSS2-ERG fusion nucleic acid comprising a fusion between a TMPRSS2 gene, or a portion thereof, and an ERG gene, or a portion thereof, or to a wild-type counterpart. In some embodiments, the kit is for use according to any method of detecting gene alterations in nucleic acid molecules known in the art or described herein, such as sequencing, next-generation sequencing, PCR, in situ hybridization methods, a nucleic acid hybridization assay, an amplification-based assay, a PCR-RFLP assay, real-time PCR, sequencing, a screening analysis, FISH, spectral karyotyping, MFISH, comparative genomic hybridization, in situ hybridization, sequence-specific priming (SSP) PCR, HPLC, or mass-spectrometric genotyping. In some embodiments, a kit provided herein further comprises instructions for detecting one or more mutations in an SPOP gene and/or a TMPRSS2-ERG fusion nucleic acid comprising a fusion between a TMPRSS2 gene, or a portion thereof, and an ERG gene, or a portion thereof, e.g., using one or more oligonucleotides, primers, probes or baits of the present disclosure.

Also provided herein are kits for detecting a polypeptide of the disclosure, e.g., a polypeptide encoded by a mutated SPOP gene comprising one or more mutations, and/or a TMPRSS2-ERG fusion polypeptide encoded by a TMPRSS2-ERG fusion nucleic acid comprising a fusion between a TMPRSS2 gene, or a portion thereof, and an ERG gene, or a portion thereof. In some embodiments, a kit provided herein comprises a reagent (e.g., one or more antibodies of the present disclosure) for detecting a polypeptide encoded by a gene comprising one or more alterations of the disclosure. In some embodiments, the kit comprises a reagent (e.g., one or more antibodies of the present disclosure) for detecting the wild-type counterparts of a polypeptide encoded by a gene comprising one or more alterations of the disclosure. In some embodiments, the reagent comprises one or more antibodies of the present disclosure capable of binding to a polypeptide encoded by a gene comprising one or more alterations of the disclosure, or to wild-type counterparts of the polypeptide. In some embodiments, the reagent comprises one or more antibodies of the present disclosure capable of distinguishing a polypeptide encoded by a gene comprising one or more alterations of the disclosure from wild-type counterparts of the polypeptide. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), or hyperdiffusion chromatography). In some embodiments, the kit further comprises instructions for detecting a polypeptide encoded by a gene comprising one or more alterations of the disclosure, e.g., using one or more antibodies of the present disclosure.

### IV. Exemplary Embodiments

The following exemplary embodiments are representative of some aspects of the invention:

Embodiment 1: A method of identifying an individual having a metastatic hormone-sensitive prostate cancer (mHSPC) or a non-metastatic castration-resistant prostate cancer (nmCRPC) who may benefit from a treatment comprising hormonal therapy, the method comprising detecting in a sample from the individual: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, wherein the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising the hormonal therapy.

Embodiment 2: A method of selecting a treatment for an individual having metastatic hormone-sensitive prostate cancer (mHSPC) or a non-metastatic castration-resistant prostate cancer (nmCRPC), the method comprising detecting in a sample from the individual: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, wherein the presence of the one or more mutations in the SPOP gene, or the SPOP polypeptide encoded by the mutated SPOP gene, and/or the TMPRSS2-ERG fusion nucleic acid identifies the individual as one who may benefit from the treatment comprising a hormonal therapy.

Embodiment 3: A method of identifying one or more treatment options for an individual having a metastatic hormone-sensitive prostate cancer (mHSPC) or a non-metastatic castration-resistant prostate cancer (nmCRPC), the method comprising: (a) detecting in a sample from the individual (i) one or more mutations in an SPOP gene, or an SPOP polypeptide encoded by the mutated SPOP gene, and/or (ii) a TMPRSS2-ERG fusion nucleic acid molecule comprising a fusion between a TMPRSS2 gene, or a portion thereof, and an ERG gene, or a portion thereof; and (b) generating a report comprising one or more treatment options identified for the individual based at least in part on detection of the one or more mutations in the SPOP gene, or the SPOP polypeptide encoded by the mutated SPOP gene, and/or the TMPRSS2-ERG fusion nucleic acid molecule in the sample, wherein the one or more treatment options comprise a hormonal therapy.

Embodiment 4: A method of selecting a treatment for an individual having a metastatic hormone-sensitive prostate cancer (mHSPC) or a non-metastatic castration-resistant prostate cancer (nmCRPC), comprising acquiring knowledge of (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, in a sample from the individual, wherein responsive to said knowledge, the individual is classified as a candidate to receive a treatment comprising a hormonal therapy.

Embodiment 5: A method of treating or delaying progression of metastatic hormone-sensitive prostate cancer (mHSPC) or non-metastatic castration-resistant prostate cancer (nmCRPC), comprising: (a) acquiring knowledge of (i) one or more mutations in an SPOP gene, or an SPOP polypeptide encoded by the mutated SPOP gene, and/or (ii) a TMPRSS2-ERG fusion nucleic acid molecule comprising a fusion between a TMPRSS2 gene, or a portion thereof, and an ERG gene, or a portion thereof, in a sample from an individual; and (b) administering to the individual an effective amount of a hormonal therapy responsive to said knowledge.

Embodiment 6: A method of predicting survival of an individual having a metastatic hormone-sensitive prostate cancer (mHSPC) or a non-metastatic castration-resistant prostate cancer (nmCRPC), comprising acquiring knowledge of: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, in a sample from the individual, wherein responsive to said knowledge, the individual is predicted to have longer survival when treated with an a hormonal therapy, as compared to an individual whose cancer does not comprise the one or more mutations in the SPOP gene, or the SPOP polypeptide encoded by the mutated SPOP gene, and/or the TMPRSS2-ERG fusion nucleic acid.

Embodiment 7: A method of predicting survival of an individual having a metastatic hormone-sensitive prostate cancer (mHSPC) or a non-metastatic castration-resistant prostate cancer (nmCRPC), comprising acquiring knowledge of: (a) one or more mutations in an SPOP gene, or an SPOP polypeptide encoded by the mutated SPOP gene, and/or (b) a TMPRSS2-ERG fusion nucleic acid molecule comprising a fusion between a TMPRSS2 gene, or a portion thereof, and an ERG gene, or a portion thereof, in a sample from the individual, wherein responsive to said knowledge, the individual is predicted to have a higher likelihood of survival when treated with an a hormonal therapy as compared to a treatment with a taxane.

Embodiment 8: The method of any one of embodiments 1-7, wherein the hormonal therapy is a novel hormonal therapy (NHT).

Embodiment 9: The method of any one of embodiments 1-8, wherein the one or more mutations in the SPOP gene are one or more of a base substitution, a short insertion/deletion (indel), a copy number alteration, or a genomic rearrangement.

Embodiment 10: The method of any one of embodiments 1-9, wherein: (a) the one or more mutations in the SPOP gene comprise one or more of a 305T>G mutation, a 397T>G mutation, a 399C>G mutation, a 393G>C mutation, a 304T>G mutation, a 374T>G mutation, a 398T>C mutation, a 398T>G mutation, a 259T>A mutation, a 260A>G mutation, a 304T>A mutation, a 356G>A mutation, a 375T>G mutation, a 389A>T mutation, a 391T>C mutation, a 391T>G mutation, a 393G>T mutation, a 397T>A mutation, a 399C>A mutation, or any combination thereof; and/or (b) the one or more mutations in the SPOP gene result in one or more amino acid substitutions in an SPOP polypeptide encoded by the mutated SPOP gene, wherein the one or more amino acid substitutions correspond to one or more of R45W, R70*, Y87C, Y87N, Y87S, Y87F, Y87D, F102C, F102V, F102S, F102I, F102L, F104V, F102Y, F104C, F104S, F104I, A116G, M117V, S119N, F125I, F125V, F125L, F125C, K129E, D130V, D130N, W131G, W131C, W131R, W131L, W131S, F133L, F133V, F133I, F133C, F133S, K134N, A277V, and L282R, or any combination thereof.

Embodiment 11: The method of any one of embodiments 1-10, wherein the one or more mutations in the SPOP gene comprise one or more of a 305T>G mutation, a 397T>G mutation, a 399C>G mutation, a 393G>C mutation, a 304T>G mutation, a 374T>G mutation, a 398T>C mutation, a 398T>G mutation, a 259T>A mutation, a 260A>G mutation, a 304T>A mutation, a 356G>A mutation, a 375T>G mutation, a 389A>T mutation, a 391T>C mutation, a 391T>G mutation, a 393G>T mutation, a 397T>A mutation, a 399C>A mutation, or any combination thereof.

Embodiment 12: The method of any one of embodiments 1-11, wherein the one or more mutations in the SPOP gene result in decreased activity of an SPOP polypeptide encoded by the mutated SPOP gene.

Embodiment 13: The method of any one of embodiments 1-12, wherein the SPOP polypeptide encoded by the mutated SPOP gene has decreased activity as compared to an SPOP polypeptide encoded by an SPOP gene that does not comprise the one or more mutations.

Embodiment 14: The method of any one of embodiments 1-13, wherein the SPOP polypeptide encoded by the mutated SPOP gene is oncogenic.

Embodiment 15: The method of any one of embodiments 1-14, wherein the SPOP polypeptide encoded by the mutated SPOP gene promotes cancer cell survival, angiogenesis, cancer cell proliferation, or any combination thereof.

Embodiment 16: The method of any one of embodiments 1-15, wherein the TMPRSS2-ERG fusion nucleic acid molecule encodes a TMPRSS2-ERG fusion polypeptide.

Embodiment 17: The method of embodiment 16, wherein the TMPRSS2-ERG fusion polypeptide encoded by the TMPRSS2-ERG fusion nucleic acid molecule is oncogenic.

Embodiment 18: The method of 16, wherein the TMPRSS2-ERG fusion polypeptide encoded by the TMPRSS2-ERG fusion nucleic acid molecule promotes cancer cell survival, angiogenesis, cancer cell proliferation, and any combination thereof.

Embodiment 19: The method of any one of embodiments 1-18, wherein the treatment further comprises administering to the individual an anti-androgen agent.

Embodiment 20: The method of any one of embodiments 1-19, wherein the hormonal therapy is a second generation hormonal therapy.

Embodiment 21: The method of any one of embodiments 1-20, wherein the hormonal therapy comprises one or more of abiraterone, enzalutamide, apalutamide, and darolutamide, or any combination thereof.

Embodiment 22: The method of any one of embodiments 1-21, wherein the hormonal therapy treatment further comprises an androgen deprivation therapy (ADT).

Embodiment 23: The method of embodiment 22, wherein the ADT comprises one or more of a orchiectomy, a LHRH agonist, a LHRH antagonist, an anti-androgen agent, an estrogen, and an androgen synthesis inhibitor, or any combination thereof.

Embodiment 24: The method of embodiment 23, wherein the orchiectomy is a bilateral orchiectomy.

Embodiment 25: The method of embodiment 23, wherein the LHRH agonist is goserelin acetate, histrelin acetate, leuprolide acetate, or triptorelin acetate.

Embodiment 26: The method of embodiment 23, wherein the LHRH antagonist is degarelix.

Embodiment 27: The method of embodiment 23, wherein the anti-androgen agent is bicalutamide, flutamide, nilutamide, enzalutamide, apalutamide, or darolutamide.

Embodiment 28: The method of embodiment 23, wherein the estrogen is diethylstilbestrol.

Embodiment 29: The method of embodiment 23, wherein the androgen synthesis inhibitor is abiraterone or ketoconazole

Embodiment 30: The method of any one of embodiments 1-29, wherein the treatment does not comprise a chemotherapy.

Embodiment 31: The method of embodiment 30, wherein the chemotherapy is a taxane.

Embodiment 32: The method of embodiment 31, wherein the taxane is docetaxel, cabazitaxel, or paclitaxel.

Embodiment 33: The method of any one of embodiments 1-32, wherein the mHSPC is a de novo mHSPC.

Embodiment 34: The method of any one of embodiments 1-32, wherein the mHSPC is a recurrent mHSPC.

Embodiment 35: The method of any one of embodiments 1-34, wherein the individual having a mHSPC or nmCRPC is at risk of progressing to a metastatic castration-resistant prostate cancer (mCRPC).

Embodiment 36: The method of any one of embodiments 1-35, wherein the individual has received a prior anti-cancer treatment, or is being treated with an anti-cancer treatment.

Embodiment 37: The method of embodiment 36, wherein the prior anti-cancer treatment comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Embodiment 38: The method of embodiment 37, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 39: The method of embodiment 38, wherein the nucleic acid comprises a double-stranded RNA (dsRNA), a small interfering RNA (siRNA), or a small hairpin RNA (shRNA).

Embodiment 40: The method of any one of embodiments 1-39, wherein the cancer has not been previously treated.

Embodiment 41: The method of any one of embodiments 1-40, wherein the treatment or the one or more treatment options further comprise an additional anti-cancer therapy.

Embodiment 42: The method of embodiment 41, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Embodiment 43: The method of embodiment 42, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Embodiment 44: The method of embodiment 42, wherein the nucleic acid comprises a double-stranded RNA (dsRNA), a small interfering RNA (siRNA), or a small hairpin RNA (shRNA).

Embodiment 45: The method of any one of embodiments 6-44, wherein the survival is an overall survival, a progression-free survival, a disease-free survival, an objective response rate, a time to tumor progression, a time to treatment failure, a durable complete response, a time to cancer-resistant pancreatic cancer progression, or a time to next treatment.

Embodiment 46: The method of any one of embodiments 1-45, wherein the sample comprises a tissue biopsy sample or a liquid biopsy sample.

Embodiment 47: The method of embodiment 46, wherein the sample is a tissue biopsy and comprises a tumor biopsy, tumor specimen, or circulating tumor cells.

Embodiment 48: The method of embodiment 46, wherein the sample is a liquid biopsy sample and comprises blood, serum, plasma, cerebrospinal fluid, sputum, stool, urine, or saliva.

Embodiment 49: The method of any one of embodiments 1-48, wherein the sample comprises cells and/or nucleic acids from the cancer.

Embodiment 50: The method of embodiment 49, wherein the sample comprises mRNA, DNA, circulating tumor DNA (ctDNA), cell-free DNA, cell-free RNA from the cancer, or any combination thereof.

Embodiment 51: The method of embodiment 49, wherein the sample is a liquid biopsy sample and comprises circulating tumor cells (CTCs).

Embodiment 52: The method of embodiment 49, wherein the sample is a liquid biopsy sample and comprises cell-free DNA (cfDNA), circulating tumor DNA (ctDNA), or any combination thereof.

Embodiment 53: The method of any one of embodiments 1-52, further comprising obtaining the sample from the individual.

Embodiment 54: The method of any one of embodiments 1-52, wherein the sample comprises cancer cells.

Embodiment 55: The method of any one of embodiments 5-54, wherein the acquiring knowledge of the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid comprises detecting the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid in the sample.

Embodiment 56: The method of any one of embodiments 1-55, wherein the one or more mutations in the SPOP gene and/or the TMPRSS2-ERG fusion nucleic acid are detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Embodiment 57: The method of embodiment 56, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Embodiment 58: The method of embodiment 56 or embodiment 57, wherein the sequencing comprises: (a) providing a plurality of nucleic acid molecules obtained from the sample, wherein the plurality of nucleic acid molecules comprises a mixture of tumor nucleic acid molecules and non-tumor nucleic acid molecules; (b) optionally, ligating one or more adapters onto one or more nucleic acid molecules from the plurality of nucleic acid molecules; (c) amplifying nucleic acid molecules from the plurality of nucleic acid molecules; (d) optionally, capturing nucleic acid molecules from the amplified nucleic acid molecules, wherein the captured nucleic acid molecules are captured from the amplified nucleic acid molecules by hybridization to one or more bait molecules; and (e) sequencing, by a sequencer, the captured nucleic acid molecules to obtain a plurality of sequence reads corresponding to one or more genomic loci within a subgenomic interval in the sample.

Embodiment 59: The method of embodiment 58, wherein the adapters comprise one or more of amplification primer sequences, flow cell adapter hybridization sequences, unique molecular identifier sequences, substrate adapter sequences, or sample index sequences.

Embodiment 60: The method of embodiment 58 or embodiment 59, wherein amplifying nucleic acid molecules comprises performing a polymerase chain reaction (PCR) technique, a non-PCR amplification technique, or an isothermal amplification technique.

Embodiment 61: The method of any one of embodiments 58-60, wherein the one or more bait molecules comprise one or more nucleic acid molecules, each comprising a region that is complementary to a region of a captured nucleic acid molecule.

Embodiment 62: The method of embodiment 61, wherein the one or more bait molecules each comprise a capture moiety.

Embodiment 63: The method of embodiment 62, wherein the capture moiety is biotin.

Embodiment 64: The method of any one of embodiments 1-63, wherein the SPOP polypeptide encoded by the mutated SPOP gene and/or the TMPRSS2-ERG fusion polypeptide encoded by the TMPRSS2-ERG fusion nucleic acid are detected in the sample by one or more of: immunoblotting, enzyme linked immunosorbent assay (ELISA), immunohistochemistry, or mass spectrometry.

Embodiment 65: The method of any one of embodiments 1-64, wherein the individual is a human.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: SPOP Mutations as a Predictive Biomarker for Enhanced Benefit with Novel Hormonal Therapy (NHT) Over Docetaxel in Patients with De Novo Metastatic Castration-Sensitive Prostate Cancer (Dn-mCSPC)

This Example describes the association of SPOP mutations and TMPRSS2-ERG rearrangements in response to novel hormonal therapy (NHT) in patients with metastatic hormone-sensitive prostate cancer (mHSPC), also known as metastatic castration-sensitive prostate cancer (mCSPC).

### Methods

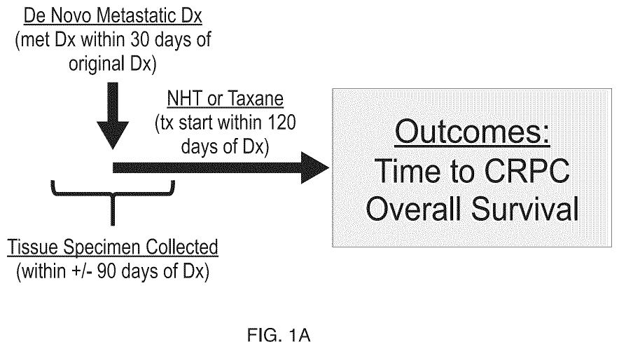

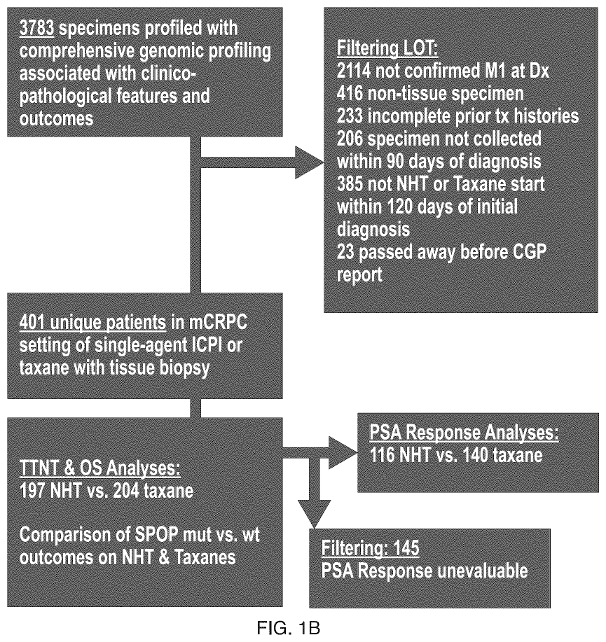

A cohort of 401 patients with metastatic hormone-sensitive prostate cancer was obtained from a de-identified clinic-genomic database (CGDB). Cohort selection was based on patients that had a tissue biopsy taken within 90 days of diagnosis of mHSPC, and initiation of treatment with NHT (abioraterone, apalutamide, or enzalutamide) or with docetaxel, in combination with androgen deprivation therapy (ADT) within 120 days of diagnosis (FIG. 1A). The time to castration-resistant prostate cancer (CRPC) and overall survival (OS), indexed from metastatic diagnosis, were evaluated using a Cox proportional hazards model adjusted for the level of prostate-specific antigen (PSA) at metastatic diagnosis as well as the ECOG performance score. The OS risk intervals were left truncated to the date of comprehensive genomic profiling reporting to account for immortal time.

Additionally, treatment interaction models were also constructed after adjusting for PSA levels, ECOG performance scores, treatment, biomarker, and interaction terms.

### Results

Of 401 patients in the cohort, 197 received NHT and 204 docetaxel. Of the cohort, 33 (9%) were found to have SPOP mutations (FIG. 1B). Table 1 is a summary of mutations in the SPOP gene identified in the patient cohort, while Table 2 summarizes the predicted amino acid substitutions to the encoded SPOP polypeptide.

TABLE 1

| | Number of |
|---|---|
| SPOP gene mutation | cases |
| 305T > G | 7 |
| 397T > G | 5 |
| 399C > G | 4 |
| 393G > C | 3 |
| 304T > G | 2 |
| 374T > G | 2 |
| 398T > C | 2 |
| 398T > G | 2 |
| 259T > A | 1 |
| 260A > G | 1 |
| 304T > A | 1 |
| 356G > A | 1 |
| 375T > G | 1 |
| 389A > T | 1 |
| 391T > C | 1 |
| 391T > G | 1 |
| 393G > T | 1 |
| 397T > A | 1 |
| 399C > A | 1 |

TABLE 2
Predicted SPOP polypeptide amino acid substitution
F133L
F133V
F102C
F102V
W131G
Y87C
Y87N
F133C
W131C
K129E
F133S
Y87S
F125V
F133I
F104V
F125L
W131R
F104C
S119N
K134N
R70*
F104S
F104I
R45W
M117V
L282R
A277V
D130N
F125C
Y87D
W131L
F102L
W131S
F102S
F125I
F102I
A116G
Y87F
D130V
F102Y

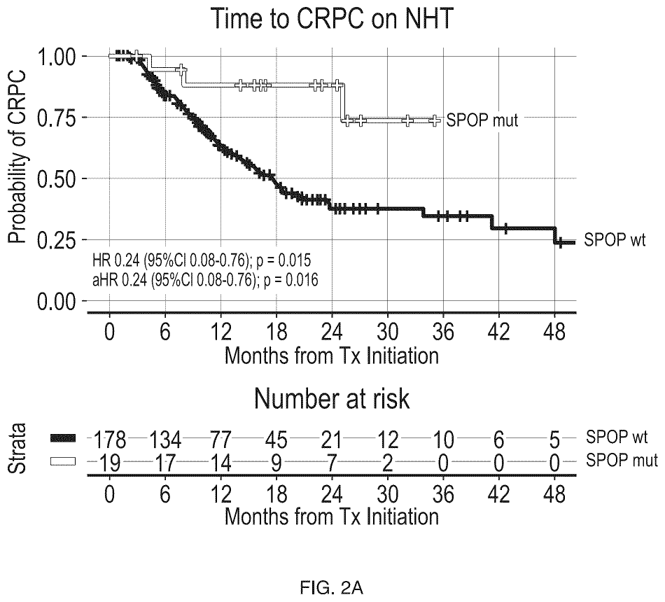

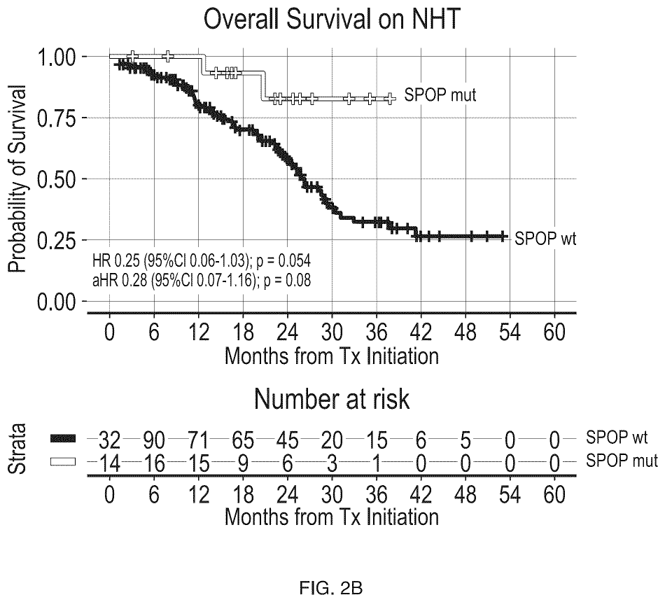

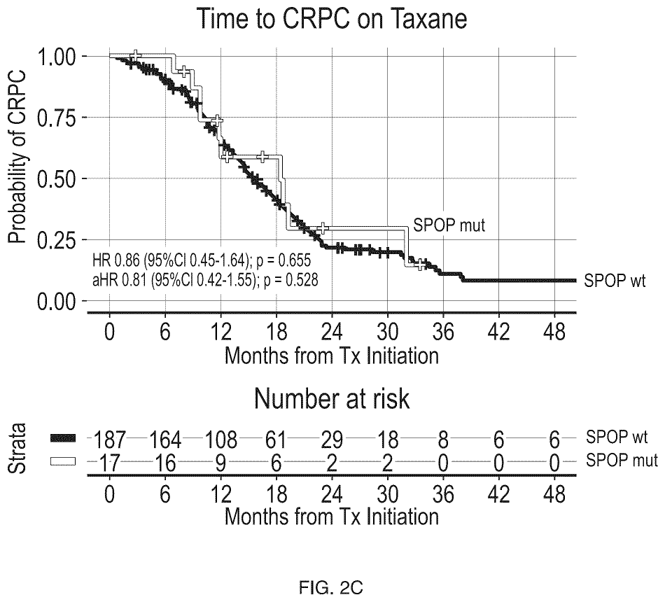

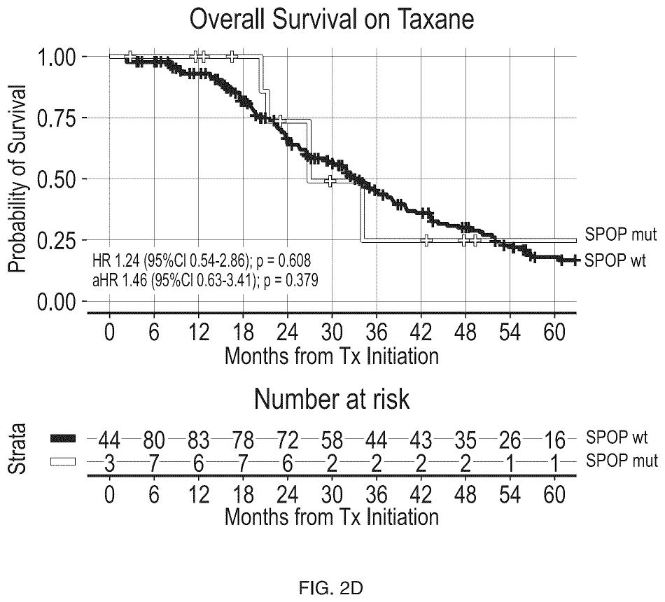

Patients with SPOP mutations receiving NHT had better time to CRPC and OS on NHT treatment than patients with a wild-type SPOP (FIGS. 2A-2B). For NHT-treated patients, the median time to CRPC was not reached in patients with SPOP mutations, but was 17.7 months for wild-type SPOP patients (adjusted hazard ratio [HR]=0.24; 95% confidence interval [CI]=0.08-0.76; p=0.016). The median OS in NHT treated patients was not reached for patients with SPOP mutations, but was 26.6 months for wild-type SPOP patients (HR=0.28; CI=0.07-1.16; p=0.08).

No significant difference in time to CRPC or OS on docetaxel treatment between patients with or without SPOP mutations (FIGS. 2C-2D). For docetaxel-treated patients, the median time to CRPC was 18.5 and 15.7 months for SPO-mutated and wild-type SPOP patients (adjusted HR=0.81; CI=0.42-1.55; p=0.53), and the median OS was 26.9 and 33.5 months for SPOP-mutated and wild-type SPOP patients, respectively (HR=1.46; CI=0.63-3.41; p=0.38).

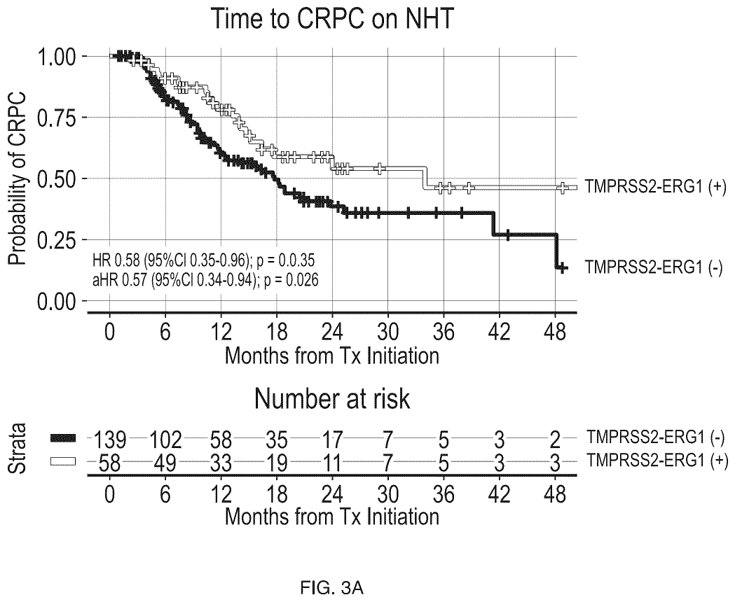

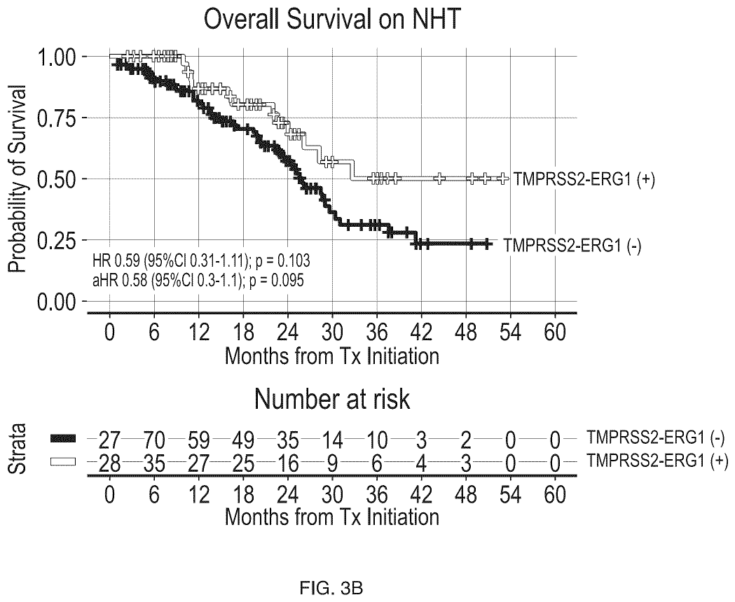

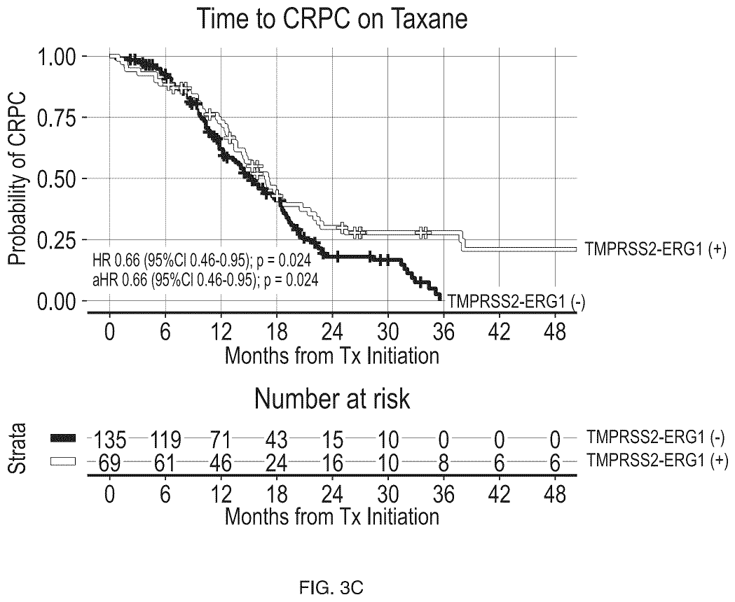

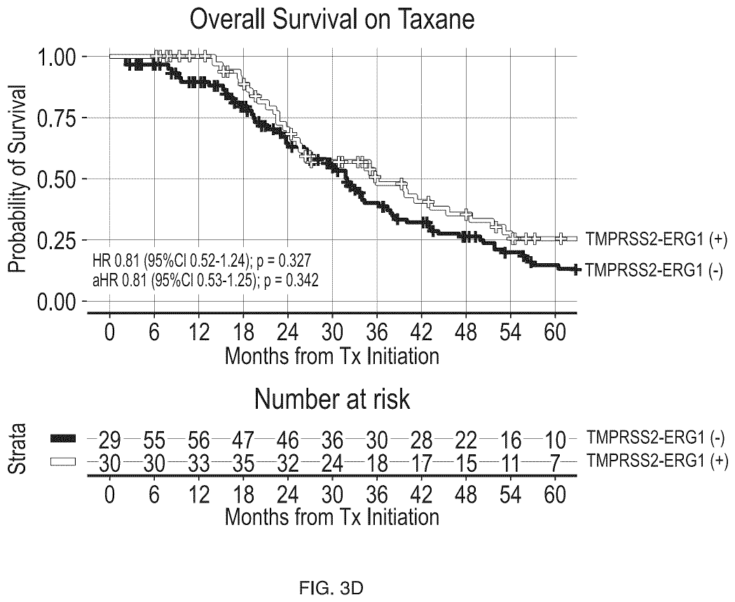

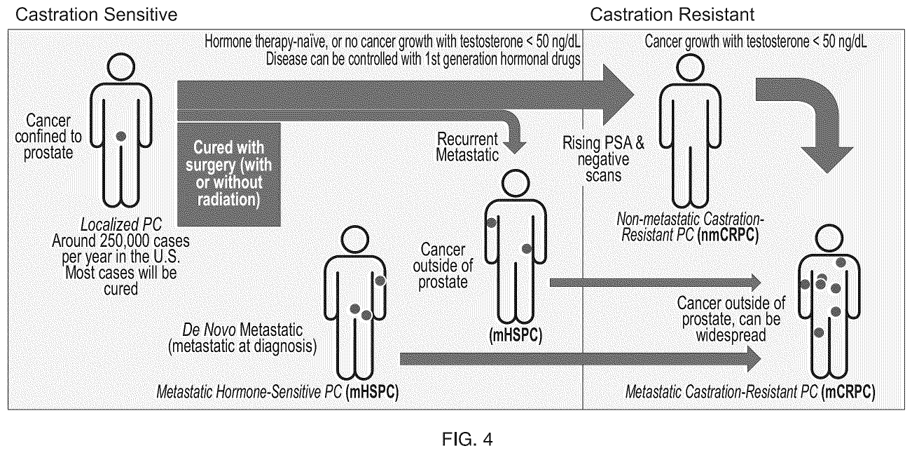

Patients with a TMPRSS2-ERG fusion receiving NHT had better time to CRPC and OS on NHT or docetaxel treatment than patients that lacked a TMPRSS2-ERG fusion (FIGS. 3A-3D).

### CONCLUSIONS

SPOP mutations could be used as a predictive biomarkers for prolonged NHT benefit over docetaxel therapy for mHSPC.

What is claimed is:

1. A method of treating or delaying progression of metastatic hormone-sensitive prostate cancer (mHSPC) or non-metastatic castration-resistant prostate cancer (nmCRPC), comprising:
(a) acquiring knowledge of
(i) one or more mutations in an SPOP gene, or an SPOP polypeptide encoded by the mutated SPOP gene, and/or
(ii) a TMPRSS2-ERG fusion nucleic acid molecule comprising a fusion between a TMPRSS2 gene, or a portion thereof, and an ERG gene, or a portion thereof,
in a sample from an individual; and
(b) administering to the individual an effective amount of a hormonal therapy responsive to said knowledge.

2. A method of predicting survival of an individual having a metastatic hormone-sensitive prostate cancer (mHSPC) or a non-metastatic castration-resistant prostate cancer (nmCRPC), comprising acquiring knowledge of:
(a) one or more mutations in an SPOP gene, or an SPOP polypeptide encoded by the mutated SPOP gene, and/or
(b) a TMPRSS2-ERG fusion nucleic acid molecule comprising a fusion between a TMPRSS2 gene, or a portion thereof, and an ERG gene, or a portion thereof,
in a sample from the individual,
wherein responsive to said knowledge, the individual is predicted to have a higher likelihood of survival when treated with a hormonal therapy as compared to a treatment with a taxane.

3. A method of identifying one or more treatment options for an individual having a metastatic hormone-sensitive prostate cancer (mHSPC) or a non-metastatic castration-resistant prostate cancer (nmCRPC), the method comprising:
(a) detecting in a sample from the individual
(i) one or more mutations in an SPOP gene, or an SPOP polypeptide encoded by the mutated SPOP gene, and/or
(ii) a TMPRSS2-ERG fusion nucleic acid molecule comprising a fusion between a TMPRSS2 gene, or a portion thereof, and an ERG gene, or a portion thereof; and
(b) generating a report comprising one or more treatment options identified for the individual based at least in part on detection of the one or more mutations in the SPOP gene, or the SPOP polypeptide encoded by the mutated SPOP gene, and/or the TMPRSS2-ERG fusion nucleic acid molecule in the sample, wherein the one or more treatment options comprise a hormonal therapy.

4. The method of claim 1, wherein the hormonal therapy is a novel hormonal therapy (NHT).

5. The method of claim 1, wherein:
(a) the one or more mutations in the SPOP gene comprise one or more of a 305T>G mutation, a 397T>G mutation, a 399C>G mutation, a 393G>C mutation, a 304T>G mutation, a 374T>G mutation, a 398T>C mutation, a 398T>G mutation, a 259T>A mutation, a 260A>G mutation, a 304T>A mutation, a 356G>A mutation, a 375T>G mutation, a 389A>T mutation, a 391T>C mutation, a 391T>G mutation, a 393G>T mutation, a 397T>A mutation, a 399C>A mutation, or any combination thereof; and/or
(b) the one or more mutations in the SPOP gene result in one or more amino acid substitutions in an SPOP polypeptide encoded by the mutated SPOP gene, wherein the one or more amino acid substitutions correspond to one or more of R45W, R70*, Y87C, Y87N, Y87S, Y87F, Y87D, F102C, F102V, F102S, F102I, F102L, F104V, F102Y, F104C, F104S, F104I, A116G, M117V, S119N, F125I, F125V, F125L, F125C, K129E, D130V, D130N, W131G, W131C, W131R, W131L, W131S, F133L, F133V, F133I, F133C, F133S, K134N, A277V, and L282R, or any combination thereof.

6. The method of claim 1, wherein the SPOP polypeptide encoded by the mutated SPOP gene has decreased activity as compared to an SPOP polypeptide encoded by an SPOP gene that does not comprise the one or more mutations; and/or wherein the SPOP polypeptide encoded by the mutated SPOP gene promotes cancer cell survival, angiogenesis, cancer cell proliferation, or any combination thereof.

7. The method of claim 1, wherein the TMPRSS2-ERG fusion nucleic acid molecule encodes a TMPRSS2-ERG fusion polypeptide.

8. The method of claim 1, wherein the hormonal therapy comprises one or more of abiraterone, enzalutamide, apalutamide, and darolutamide, or any combination thereof.

9. The method of claim 1, further comprising administering to the individual an anti-androgen agent.

10. The method of claim 1, wherein the hormonal therapy treatment further comprises an androgen deprivation therapy (ADT).

11. The method of claim 1, wherein the treatment does not comprise a taxane.

12. The method of claim 1, wherein the mHSPC is a de novo mHSPC or a recurrent mHSPC.

13. The method of claim 1, wherein the individual has received a prior anti-cancer treatment, or is being treated with an anti-cancer treatment.

14. The method of claim 1, wherein the cancer has not been previously treated.

15. The method of claim 1, further comprising administering to the individual an additional anti-cancer therapy.

16. The method of claim 1, wherein the sample comprises a tissue biopsy sample or a liquid biopsy sample, and wherein the sample comprises cells and/or nucleic acids from the cancer.

17. The method of claim 1, wherein the acquiring knowledge of the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid comprises detecting the one or more mutations in the SPOP gene, or the SPOP polypeptide encoded by the mutated SPOP gene and/or the TMPRSS2-ERG fusion nucleic acid or the TMPRSS2-ERG fusion polypeptide encoded by the TMPRSS2-ERG fusion nucleic acid in the sample.

18. The method of claim 17, wherein the one or more mutations in the SPOP gene and/or the TMPRSS2-ERG fusion nucleic acid are detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

19. The method of claim 18, wherein the sequencing comprises:
(a) providing a plurality of nucleic acid molecules obtained from the sample, wherein the plurality of nucleic acid molecules comprises a mixture of tumor nucleic acid molecules and non-tumor nucleic acid molecules;
(b) optionally, ligating one or more adapters onto one or more nucleic acid molecules from the plurality of nucleic acid molecules;
(c) amplifying nucleic acid molecules from the plurality of nucleic acid molecules;
(d) optionally, capturing nucleic acid molecules from the amplified nucleic acid molecules, wherein the captured nucleic acid molecules are captured from the amplified nucleic acid molecules by hybridization to one or more bait molecules; and
(e) sequencing, by a sequencer, the captured nucleic acid molecules to obtain a plurality of sequence reads corresponding to one or more genomic loci within a subgenomic interval in the sample.

20. The method of claim 1, wherein the individual is a human.
